(19) United States
(12) Patent Application Publication
(10) Pub. No.: US 2011/0053020 A1
(43) Pub. Date: Mar. 3, 2011

(54) CATALYSTS AND RELATED METHODS

(75) Inventors: M. Grant Norton, Pullman, WA (US); David N. McIlroy, Moscow, ID (US)

(73) Assignees: WASHINGTON STATE UNIVERSITY RESEARCH FOUNDATION, Pullman, WA (US); IDAHO RESEARCH FOUNDATION, INC., Moscow, ID (US)

(21) Appl. No.: 12/513,524

(22) PCT Filed: Nov. 7, 2008
(86) PCT No.: PCT/US08/82884

§ 371 Date: May 5, 2009

Related U.S. Application Data
(60) Provisional application No. 61/031,273, filed on Feb. 25, 2008.
(60) Provisional application No. 60/986,957, filed on Nov. 9, 2007.

(51) Int. Cl. H01M 8/06; F02B 43/00; C01B 3/02; C01B 3/16; C01B 3/26; B01J 23/00; B01J 21/08; B01J 27/24; B01J 23/52; B01J 23/50; B01J 23/42; B01J 23/745; B01J 23/755; B01J 23/75; B01J 23/72; B01J 23/44; B01J 23/06; B01J 21/06; C07C 5/03; B82Y 30/00; B82Y 40/00

(52) U.S. Cl. 429/425; 123/3; 502/1; 423/648.1; 423/655; 423/651; 502/300; 502/240; 502/200; 502/344; 502/347; 502/339; 502/338; 502/337; 502/325; 502/345; 502/243; 502/262; 502/343; 502/350; 502/253; 502/244; 502/261; 502/258; 502/259; 502/260; 585/250; 977/948; 977/775; 977/700; 977/840

(57) ABSTRACT

Nanostructured catalysts and related methods are described. The nanostructured catalysts have a hierarchical structure that facilitates modification of the catalysts for use in particular reactions. Methods for generating hydrogen from a hydrogen-containing molecular species using a nanostructured catalyst are described. The hydrogen gas may be collected and stored, or the hydrogen gas may be collected and consumed for the generation of energy. Thus, the methods may be used as part of the operation of an energy-consuming device or system, e.g., an engine or a fuel cell. Methods for storing hydrogen by using a nanostructured catalyst to react a dehydrogenated molecular species with hydrogen gas to form a hydrogen-containing molecular species are also described.

### CROSS-REFERENCE TO RELATED APPLICATIONS

This application claims the benefit of priority to U.S. Provisional Patent Application Ser. No. 60/986,957, entitled “Method for Efficient Evolution of Hydrogen,” filed Nov. 9, 2007 and U.S. Provisional Patent Application Ser. No. 61/031,273, entitled “Catalysts and Related Methods,” filed Feb. 25, 2008, each of which is incorporated by reference herein in its entirety.

### FIELD

Described herein are catalytic materials that comprise nanostructures at least partially coated with metal-containing nanoparticles. The catalytic materials may be used in a variety of catalysis applications, including dehydrogenation, hydrogenation, and polymerization reactions.

### BACKGROUND

In general, it is desirable for a catalyst to be reusable, e.g., to reduce costs, processing steps, and environmental waste. Further, it is desirable to reduce or eliminate the need for toxic or corrosive agents such as acids that have historically been used in some catalysis reactions. The catalytic properties of some nanostructures have been investigated for some reactions, such as in oxidation reactions. See, e.g., Idakiev et al., Appl. Catal. A, 281 (2005), 149; Xu et al., J. Catal. 237 (2006), 426; Bennett et al., Faraday Discuss., 114 (1999), 267; Sun et al., Adv. Mater., 17 (2005), 2993; Chen et al., Adv. Mater., 17 (2005), 582; Bogue, Sensor Review, 24 (2004), 253; Wu et al., Sensors and Actuators B, 115 (2006), 198; Jiang et al., Nano Lett., 2 (2002), 1333. In one example, nanocrystalline zinc oxide has been studied as a catalyst for the formation of tetrazoles. See Kantam, et al., Adv. Synth. Catal., 347 (2005), 1212.

Among various alternative energy strategies, an energy infrastructure using hydrogen may be a promising approach that offers advantages in certain circumstances. Hydrogen is the third most abundant element on the earth's surface and offers the highest energy density per unit weight of any known fuel. The heat of combustion for hydrogen is 125 MJ/kg, about 3 times higher than that of gasoline (43 MJ/kg). Hydrogen is the lightest element; one gram of hydrogen occupies a volume of 11 liters at atmospheric pressure and therefore would have added efficiency by reducing fuel storage weight relative to petroleum fuels. It is a renewable resource and can be produced from a variety of sources, such as steam reforming of natural gas, electrolysis of water, and bio-inspired photosynthesis of CO₂, H₂O and sunlight to H₂ and O₂. Hydrogen use may offer a strategy to reduce greenhouse gas emissions where the byproduct is water, e.g., in a hydrogen combustion engine or a hydrogen fuel cell.

One challenge in the use of hydrogen as a fuel, e.g., for automobiles, is storing hydrogen. Hydrogen gas stored at a high pressure may have a high potential for ignition, making it difficult to use safely in consumer applications. Hydrogen may be liquefied and stored cryogenically; however, cryogenic cooling may require the expenditure of undesired amounts of energy. In some cases, hydrogen may be stored in molecular complexes, e.g., metals that can react with hydrogen to form metal hydrides. Catalytic methods can then be used to trigger release of the hydrogen from the molecular complexes. However, high temperatures may be required to release hydrogen from metal hydrides.

A need exists for improved catalyst systems and methods for catalyzing reactions such as hydrogenation, dehydrogenation, oxidation, and polymerization. For example, a need exists for improved systems and methods for storing hydrogen and controllably releasing the hydrogen for use as fuel.

### SUMMARY

Nanostructured catalysts are described herein. In some variations, the catalysts comprise a disordered array of nanostructures and a plurality of metal-containing (e.g., metals, metal alloys, metal oxides, and the like) nanoparticles attached to the nanostructures to form metallized nanostructures. An average cross-sectional dimension of the nanoparticles is at most half an average cross-sectional dimension of the nanostructures. The disordered array of metallized nanostructures provides a macroporous network comprising accessible catalytic sites that can adsorb one or more reactants in a reaction to be catalyzed.

The catalytic activity and/or selectivity of the nanostructured catalysts may be modified to tune the catalyst for use in catalyzing a particular reaction. In some variations, a catalytic activity and/or selectivity of a catalyst is modified or tuned by selecting the size of accessible catalytic sites in the macroporous network. For example, for certain reactions, a catalyst comprising a macroporous network with relatively fewer accessible catalytic sites but having relatively large size of accessible catalytic sites may be preferred over a catalyst comprising a macroporous network with relatively more accessible catalytic sites but having relatively small size of accessible catalytic sites.

In some variations, the size of accessible catalytic sites may be tuned by adjusting the configurations (e.g., shapes or structure types and dimensions) of the nanostructures in the disordered array. In the disordered array, the nanostructures may be selected from the group consisting of nanowires, nanotubes, nanorods, nanosprings, and combinations thereof. In some variations, a majority of the nanostructures in the disordered array may be rod-like. In other variations, a majority of the nanostructures may be coils (i.e. nanosprings). The nanostructures in a disordered array may have similar structure types (e.g., essentially all nanosprings) or to different structure types (e.g., a mixture of nanowires and nanosprings as may be grown by changing growth conditions, so that nanosprings grow upon a mat of nanowires already laid down for instance, or vice versa). Further, the nanostructures in a disordered array may have similar dimensions (e.g., having similar cross-sectional dimensions or lengths) or different dimensions (e.g., a mixture of relatively thick nanostructures with relatively thin nanowires or a mixture of relatively long nanostructures with relatively short nanostructures). Alternatively to or in addition to varying the nanostructure configurations in a disordered array, the size of accessible catalytic sites may be tuned by selecting a density of nanostructures in the disordered array. In some variations, both the configurations and density of the nanostructures in the disordered array may be varied to adjust a size of accessible catalytic sites.

In addition, the catalytic activity and/or selectivity of the nanostructured catalysts may be modified by selecting at least one of a metal contained in the nanoparticles and the average cross-sectional dimension of the nanoparticles. In some variations, the compositions of the nanoparticles and nanostructures may be selected so that an electronic interaction between the nanostructures and the nanoparticles affects the activity of the catalyst.

In the catalysts, the nanostructures may have any suitable composition. For example, in some variations, the nanostructures may comprise SiO₂. In other variations, the nanostructures may comprise a semiconductor, such as a wide bandgap semiconductor. Non-limiting examples of semiconducting materials that may be used to form nanostructures include GaN, SiC, TiO₂, ZnO, AlN, copper oxide such as copper (I) oxide, copper (II) oxide or a mixture thereof, and cobalt oxide such as cobalt (II) oxide, cobalt (III) oxide, cobalt (IV) oxide, cobalt (II,III) oxide, cobalt (II,IV) oxide or a mixture thereof.

As stated above, at least one of the metals contained in the nanoparticles and an average dimension of the nanoparticles is selected to catalyze the particular reaction at issue. In certain variations, nanoparticles may be selected to contain a metal from the group consisting of Au, Ag, Pt, Pd, Cu, Fe, Rh, Ru, Ni, Co and alloys and combinations thereof. For example, palladium nanoparticles may be selected to catalyze an alkyne hydrogenation. In other variations, the nanoparticles may comprise a metal oxide, e.g., zinc oxide, titanium dioxide, copper oxide such as copper (I) oxide, copper (II) oxide or a mixture thereof, or cobalt oxide such as cobalt (II) oxide, cobalt (III) oxide, cobalt (IV) oxide, cobalt (II,III) oxide, cobalt (II,IV) oxide or a mixture thereof. For example, titanium dioxide nanoparticles may be selected to catalyze polymerization of ethylene or propylene. The size of the nanoparticles may be selected to control a rate of catalysis in certain applications. In certain variations of the catalysts, the nanoparticles may have a cross-sectional dimension in a range from about 1 nm to about 100 nm, e.g., about 1 nm to about 5 nm, about 1 nm to about 15 nm, about 2 nm to about 15 nm, or about 5 nm to about 100 nm.

In some cases, the distribution of nanoparticles on the nanostructures may be adjusted to modify the activity of the catalyst for certain reactions. In some variations, the nanoparticles are distributed on the nanostructures such that a majority of the nanoparticles are generally isolated from each other. In some variations, the nanoparticles may be distributed on the nanostructures such that there is physical contact between at most about 30% of the nanoparticles.

Any composition and configuration of nanostructure described herein may be combined with any composition and size of nanoparticle described herein. For example, some catalysts may comprise silica nanostructures (e.g., nanosprings or nanowires) with gold, palladium, platinum or nickel nanoparticles. Some catalysts may comprise nanostructures (e.g., nanowires) consisting essentially of GaN with gold nanoparticles attached thereto. In some cases, a nanostructure (e.g., a GaN nanowire or a SiO₂ nanospring) may be coated with a metal oxide (e.g., zinc oxide, titanium dioxide, copper oxide such as copper (I) oxide, copper (II) oxide or a mixture thereof, or cobalt oxide such as cobalt (II) oxide, cobalt (III) oxide, cobalt (IV) oxide, cobalt (II,III) oxide, cobalt (II,IV) oxide or a mixture thereof) and metal-containing nanoparticles (e.g., Au, Ag, Cu, Pd, Pt, Rh, Ru, Fe, Ni, Co, or alloys or combinations thereof) may be attached to the metal oxide. As stated above, in some variations, the compositions of the nanoparticles and nanostructures may be selected so that an electronic interaction between the nanostructures and the nanoparticles affects the activity of the catalyst. For example, an electronic interaction between a wide bandgap semiconductor (e.g., GaN) nanostructure and a metal nanoparticle (e.g., Au) attached thereto may enhance catalytic activity.

Methods for catalyzing a reaction using the nanostructured catalysts are disclosed herein. The methods comprise selecting a size of accessible catalytic sites in a macroporous network so as to adsorb or bind one or more reactants of the reaction, where the macroporous network is formed from a disordered array of nanostructures and a plurality of metal-containing nanoparticles attached to the nanostructures. The methods also comprise selecting at least one of the metal contained in the nanoparticles and an average cross-sectional dimension of the nanoparticles to catalyze the reaction. In certain methods, the compositions of the nanoparticles and nanostructures may be selected so that an electronic interaction between the nanostructures and the nanoparticles affects the catalysis of the reaction.

In some variations of the methods, configurations of the nanostructures within the disordered array can be varied to change the size of accessible catalytic sites. For example, nanostructure configurations may be varied among nanorods, nanosprings, nanowires, nanotubes and combinations thereof. Further, cross-sectional dimensions and lengths of the nanostructures can be varied. Alternatively to or in addition to varying nanostructure configurations, nanostructure density within the disordered array can be varied to affect the size of accessible catalytic sites.

Any suitable nanostructure composition may be used in the methods. For example, nanostructures comprising SiO₂ may be used in some variations. In certain variations, nanostructures comprising a semiconductor material, e.g., a wide bandgap semiconductor may be used. Non-limiting examples of semiconducting materials that may be used include GaN, SiC, TiO₂, ZnO, AlN, copper oxide such as copper (I) oxide, copper (II) oxide or a mixture thereof, and cobalt oxide such as cobalt (II) oxide, cobalt (III) oxide, cobalt (IV) oxide, cobalt (II,III) oxide, cobalt (II,IV) oxide or a mixture thereof.

Any suitable composition and dimension of metal-containing nanoparticles may be used in the methods. In certain variations, nanoparticles comprising a metal selected from the group consisting of Au, Ag, Cu, Fe, Ni, Pt, Pd, Rh, Ru, Co, and alloys and combination thereof may be used. In some cases, nanoparticles comprising a metal oxide may be used, e.g., zinc oxide, titanium dioxide, copper oxide such as copper (I) oxide, copper (II) oxide or a mixture thereof, or cobalt oxide such as cobalt (II) oxide, cobalt (III) oxide, cobalt (IV) oxide, cobalt (II,III) oxide, cobalt (II,IV) oxide or a mixture thereof. In some cases, the nanoparticles and the nanostructures may be selected to have an electronic interaction so as to affect catalytic activity. For example, some methods may employ GaN nanostructures with Au nanoparticles attached in the macroporous network. In some cases, the distribution of nanoparticles on the nanostructures may be varied to catalyze the reaction. For example, in some cases, the nanoparticles may be distributed on the nanostructures so that a majority of the nanoparticles are generally isolated from each other. In certain instances, the nanoparticles may be distributed on the nanostructures such that there is physical contact between at most about 30% of the nanoparticles.

The methods may be used to catalyze a variety of different types of reactions. For example, in some variations, the methods may be used to catalyze a dehydrogenation reaction. to Other methods may be used to catalyze a hydrogenation reaction. For example, palladium nanoparticles on silica nanostructures may in some instances be used to catalyze hydrogenation (e.g., partial hydrogenation) of alkynes. The methods may be used to catalyze a polymerization reaction, e.g., a macroporous network comprising silica nanostructures coated with TiO₂ nanoparticles may be useful in catalyzing ethylene or propylene polymerization reactions.

Other variations of catalyst devices are described herein. The devices comprise a disordered array of nanostructures, the nanostructures comprising a wide bandgap semiconductor material, and a plurality of metal-containing nanoparticles disposed on the nanostructures to form a disordered array of metallized nanostructures. The disordered array of metallized nanostructures provides a macroporous network with accessible catalytic sites. In some devices, the semiconductor material may be selected from the group consisting of GaN, SiC, TiO₂, ZnO AlN, copper oxide such as copper (I) oxide, copper (II) oxide or a mixture thereof, and cobalt oxide such as cobalt (II) oxide, cobalt (III) oxide, cobalt (IV) oxide, cobalt (II,III) oxide, cobalt (II,IV) oxide or a mixture thereof. In some devices, the metal-containing nanoparticles may comprise a metal selected from the group consisting of Au, Fe, Co, Ni, Rh, Ru, Pt, Pd, Ag and alloys and combinations thereof.

In certain variations of the catalyst devices, the nanoparticles may have a cross-sectional dimension in a range from about 1 nm to about 100 nm, e.g., about 1 nm to about 5 nm, about 1 nm to about 15 nm, about 2 nm to about 15 nm, or about 5 nm to about 100 nm. In some variations, the nanoparticles are distributed on the nanostructures such that a majority of the nanoparticles are generally isolated from each other. In some variations, the nanoparticles may be distributed on the nanostructures such that there is physical contact between at most about 30% of the nanoparticles.

Methods for making a catalysis device are described. These methods comprise forming a disordered array of nanostructures comprising a wide band gap semiconductor material and disposing a plurality of metal-containing nanoparticles on the nanostructures to form metallized nanostructures. The disordered array of metallized nanostructures provides a macroporous network comprising accessible catalytic sites that can adsorb or bind one or more reactants to catalyze a reaction. The methods may comprise forming nanostructures having an average cross-sectional dimension in a range from about 5 nm to about 200 nm. The semiconductor material may be selected from the group consisting of GaN, Si, SiC, TiO₂, ZnO AlN, copper oxide such as copper (I) oxide, copper (II) oxide or a mixture thereof, and cobalt oxide such as cobalt (II) oxide, cobalt (III) oxide, cobalt (IV) oxide, cobalt (II,III) oxide, cobalt (II,IV) oxide or a mixture thereof. The nanoparticles may be selected from the group consisting of Au, Fe, Co, Ni, Cu, Rh, Ru, Pt, Pd and Ag. The nanoparticles may have a cross-sectional dimension in a range from about 1 nm to about 100 nm, e.g., about 1 nm to about 5 nm, about 5 nm to about 100 nm, or about 2 nm to about 15 nm.

Methods and devices for generating hydrogen gas from hydrogen-containing molecular species using nanostructured catalysts are described. The hydrogen gas thus generated may be collected and stored and/or the hydrogen gas may be collected and consumed by an energy-consuming device or system. Thus, the methods and devices in some cases may be used as part of the operation of an energy-consuming device or system, e.g., an engine, a fuel cell, or the like. Methods and devices are also described herein for storing hydrogen by using nanostructured catalysts to react dehydrogenated or spent molecular species with hydrogen gas to form corresponding hydrogen-containing molecular species from which hydrogen gas may later be catalytically released for use by an energy-consuming device or system.

Some methods for producing hydrogen comprise exposing an oxygen catalysis device to CO in an enclosure, where the oxygen catalysis device comprises a disordered array of nanostructures comprising a wide bandgap semiconductor materials and a plurality of metal-containing nanoparticles disposed on the nanostructures, evacuating CO from the enclosure, and, after evacuating CO from the enclosure, introducing a molecule comprising hydrogen and oxygen to the oxygen catalyst device to produce hydrogen. In some variations of these methods, the molecule comprising hydrogen and oxygen may comprise H₂O so that CO₂ and hydrogen is produced. In certain variations, the molecule comprising hydrogen and oxygen comprises CH₃OH. In these methods, the nanostructures in the disordered array may have a cross-sectional dimension that is in a range from about 5 nm to about 200 nm. The semiconductor material may in some variations be selected from the group consisting of GaN, Si, SiC, TiO₂, ZnO, AlN, copper oxide such as copper (I) oxide, copper (II) oxide or a mixture thereof, and cobalt oxide such as cobalt (H) oxide, cobalt (III) oxide, cobalt (IV) oxide, cobalt (II,III) oxide, cobalt (II,IV) oxide or a mixture thereof. The metal contained in the nanoparticles may be selected from a group consisting of Au, Fe, Co, Ni, Cu, Rh, Ru, Pt, Pd, Ag and alloys and combinations thereof. The nanoparticles may have an average cross-sectional dimension in a range from about 1 nm to about 100 nm, e.g., about 1 nm to about 4.5 or 5 mn, about 5 nm to about 100 nm, or about 2 nm to about 15 nm.

Other methods for generating hydrogen are described here. These methods include providing a nanostructured catalyst that comprises metal-containing nanoparticles, where the metal-containing nanoparticles are disposed on a substrate and/or on a disordered array (mesh or mat) of nanostructures, e.g., nanowires, nanotubes, nanosprings, nanorods, or a combination thereof, and reacting a compound capable of generating hydrogen and having a formula R₁—XH with the nanostructured catalyst to produce hydrogen gas. The methods may include producing R₁—X that is bound to the nanostructured catalyst and/or a dehydrogenated spent compound. In these methods, R₁ is a moiety selected from the group consisting of an alkyl, a heteroalkyl, an alkenyl, a substituted alkenyl, an alkynyl, an aryl, a heteroaryl, an alkoxy, a cycloalkyl, a heterocylic group, an alkylaryl, an arylalkyl, an arylalkenyl, an arylalkynyl, an arylene, an oxyarylene group, and combinations thereof, and X is selected from the group consisting of sulfur, oxygen and selenium. The methods may include collecting the hydrogen gas, which in some variations may comprise consuming the hydrogen gas in an engine, a fuel cell, or the like.

The nanostructures in the disordered array may have a cross-sectional dimension in a range from about 5 nm to about 100 nm, or about 5 nm to about 500 nm. The substrate and/or at least some of the nanostructures in the disordered array may, in some variations, comprise a semiconductor, e.g., a wide bandgap semiconductor. For example, in some variations, at least some of the nanostructures in the disordered array may comprise at least one of the group consisting of GaN, Si, SiC, TiO₂, ZnO, AlN, copper oxide such as copper (I) oxide, copper (II) oxide or a mixture thereof, and cobalt oxide such as cobalt (II) oxide, cobalt (III) oxide, cobalt (IV) oxide, cobalt (II,III) oxide, cobalt (II,IV) oxide or a mixture thereof. In some variations, at least some of the nanostructures in the disordered array may comprise SiO₂. In certain variations, at least some of the nanostructures in the disordered array may comprise a SiO₂ nanostructure that is at least partially coated with a single crystal and/or polycrystalline coating of a semiconductor, e.g., GaN, ZnO, TiO₂, copper oxide such as copper (I) oxide, copper (II) oxide or a mixture thereof, or cobalt oxide such as cobalt (II) oxide, cobalt (III) oxide, cobalt (IV) oxide, cobalt (II,III) oxide, cobalt (II,IV) oxide or a mixture thereof.

The metal-containing nanoparticles disposed on the substrate and/or disordered array of nanostructures may have any suitable composition, particle size distribution, and spatial distribution. For example, in some variations, the nanoparticles may have a dimension from about 2 nm to about 100 nm, from about 2 nm to about 50 nm, from about 2 nm to about 20 nm, from about 2 nm to about 15 nm, or from about 2 nm to about 10 nm. The metal-containing nanoparticles may comprise one or more metals selected from the group consisting of Au, Ag, Pt, Pd, Rh, Ru, Cu, Fe, Ni, Co, and combinations thereof. In some variations of the methods, a nanostructured catalyst may be used that comprises a disordered array of nanostructures and/or a substrate comprising GaN decorated with Au nanoparticles.

In certain variations, the nanoparticles may be distributed on the surface of a substrate and/or on the nanostructures such that the nanoparticles are generally isolated from each other. For example, there may be physical contact between at most about 10%, at most about 20%, at most about 30%, at most about 40%, or at most about 50% of the particles. If there is physical contact between particles, the contact may be such that the particles merely abut each other, but a boundary or relatively clear demarcation exists between two abutting particles. In some cases, a surface area to mass ratio of the nanoparticles on the substrate and/or nanostructures is at least about 50 m²/g.

In some variations of these methods, R₁ may be a C2-C8 alkyl, a C2-C8 heteroalkyl, a C2-C8 alkenyl, or a C2-C8 heteroalkenyl group. For example, the methods may comprise reacting a C2-C8 organothiol, e.g., 1,4-cyclohexanedithiol, with the nanostructured catalyst to produce hydrogen gas. Certain variations of the methods may comprise reacting a compound having a formula R₁—SH with the nanostructured catalyst to produce hydrogen gas.

Other methods for generating hydrogen are provided. These methods include providing a nanostructured catalyst comprising metal-containing nanoparticles disposed on a substrate and/or a disordered array (mesh or mat) of nanostructures, as described above. In these methods, a cycloalkane is reacted with nanostructured catalyst to produce hydrogen gas and a dehydrogenated molecular species bound to the nanostructured catalyst and/or a dehydrogenated spent compound. The methods include collecting the hydrogen gas, which may in some instances comprise consuming the hydrogen gas with an energy-consuming device or system, e.g., an engine, fuel cell, or the like.

In some variations of these methods, the nanoparticles in the nanostructured catalyst may comprise Pt, Pd, Ru, Rh, or a combination thereof. The cycloalkane, e.g., a liquid cycloalkane, may be selected from the group consisting of cyclohexane, methylcyclohexane, cis-decalin, and trans-decalin. The nanoparticles disposed on the substrate and/or nanostructures may have a dimension of about 2 nm to about 15 nm. In variations using a nanostructured catalyst comprising a disordered array of nanostructures, the disordered array may comprise nanostructures having dimensions of about 5 nm to about 100 nm.

Methods for storing hydrogen for use in generating energy are described herein. These methods include providing a nanostructured catalyst comprising metal-containing to nanoparticles disposed on a substrate and/or nanostructures forming a disordered array, as described above. The nanoparticles disposed on the substrate and/or disordered array of nanostructures may have a cross-sectional dimension in a range from about 2 nm to about 15 nm. If present, the disordered array may comprise nanostructures having cross-sectional dimensions of about 5 nm to about 100 nm. The methods include reacting a dehydrogenated compound with hydrogen gas in the presence of the nanostructured catalyst to produce a compound having a formula R₁—XH and collecting the compound having the formula R₁—XH. In these methods, R₁ may be a moiety selected from the group consisting of an alkyl, a heteroalkyl, an alkenyl, a substituted alkenyl, an alkynyl, an aryl, a heteroaryl, an alkoxy, a cycloalkyl, a heterocyclic group, an alkylaryl, an arylalkyl, an arylalkenyl, an arylalkynyl, an arylene group, an oxyarylene group, and combinations thereof. X is a heteroatom selected from the group consisting of sulfur, oxygen, and selenium.

In some variations of these methods, the dehydrogenated compound may be selected from the group consisting of R₁—X—X—R₁, R₂═XH, and R₃=X, wherein R₂ and R₃ are dehydrogenated relative to R₁. In certain variations, the dehydrogenated compound may be selected from the group consisting of 1,2-dithiolane, dithioparabenzoquinone, and 1,4-benzendithiol.

Other methods for storing hydrogen for use in generating energy are described. These methods also include providing a nanostructured catalyst comprising metal-containing nanoparticles disposed on substrate and/or a disordered array of nanostructures, as described above. The nanoparticles disposed on the substrate and/or nanostructures forming the disordered array may have dimensions of about 2 nm to about 15 nm. If present, the disordered array may comprise nanostructures having cross-sectional dimensions of about 5 nm to about 100 nm. These methods include reacting a dehydrogenated compound with hydrogen gas in the presence of the nanostructured catalyst to produce a cycloalkane, e.g., a liquid cycloalkane, and collecting the cycloalkane. Some variations of the methods may comprise producing cyclohexane, methylcyclohexane, cis-decalin, or trans-decalin.

### BRIEF DESCRIPTION OF THE DRAWINGS

FIG. 1 illustrates an example of a nanostructured catalyst. In this particular example, the nanostructures consist essentially of SiO₂ and the nanoparticles are Au.

FIGS. 2A-2C schematically illustrate charge depletion regions created in a semiconducting nanostructure by deposition of metal-containing nanoparticles on the surface of the nanostructure, and adsorption of a species onto the metal-containing nanoparticle-decorated nanostructure surface.

FIG. 3 illustrates some examples of hydrogen-containing molecular species that may be reacted with the nanostructured catalysts described herein to produce hydrogen gas as a source of fuel.

FIG. 4 illustrates additional examples of hydrogen-containing molecular species that may be reacted with the nanostructured catalysts described herein to produce hydrogen gas as a source of fuel.

FIGS. 5A-5C are transmission electron microscope images of Pt nanoparticles on an approximately 40 nm diameter SiO₂ nanowire, an approximately 70 nm diameter SiO₂ nanowire, and an approximately 35 nm diameter SiO₂ nanowire, respectively.

FIG. 5D shows a histogram of a particle size distribution of Pt nanoparticles formed on the nanowires as illustrated in FIGS. 5A-5C.

FIG. 6 is a transmission electron microscope image of a variation of a nanostructured catalyst comprising gold nanoparticles on a GaN nanowire.

FIGS. 7A-7B are transmission electron microscope images of a variation of a nanostructured catalyst comprising gold nanoparticles disposed on a disordered array of GaN nanowires having varying cross-sectional dimensions.

FIGS. 8A-8B are transmission electron microscope images of a variation of a nanostructured catalyst comprising gold nanoparticles on a disordered array of GaN nanowires having varying morphologies.

FIGS. 9A-9C are transmission electron microscope images of variations of nanostructured catalysts comprising ZnO coated onto SiO₂ nanosprings.

FIGS. 10A-10B are transmission electron microscope images of variations of nanostructured catalysts comprising TiO₂ coated onto SiO₂ nanosprings.

### DETAILED DESCRIPTION

The following detailed description should be read with reference to the drawings, in which identical reference numbers refer to like elements through the different figures. The drawings, which are not necessarily to scale, depict selective embodiments and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives, and uses of the invention, including what is presently believed to be the best mode of carrying out the invention. It should also be noted that, as used in this specification and the appended claims, the singular forms “a,” “an,” and “the” include plural referents unless the context clearly indicates otherwise. Reference to “about” a value or parameter herein includes (and describes) embodiments that are directed to that value or parameter per se. For example, description referring to “about X” includes description of “X.” A description referring to a “range from about X to about Y” includes description of “X” and “Y” and values between X and Y. Unless defined otherwise, all technical and scientific terms used herein have the same meaning as is commonly understood by one of ordinary skill in the art to which this invention belongs. If a definition set forth in this section is contrary to or otherwise inconsistent with a definition set forth in patents, published patent applications, and other publications that are herein incorporated by reference, the definition set forth in this section prevails over the definition that is incorporated herein by reference.

As used herein, “nanostructure” is meant to encompass any structure having at least one dimension of about 100 nm or smaller, and “nanoparticle” is meant to encompass any particle having at least one dimension of about 100 nm or smaller. By “disordered array” it is meant that the nanostructures form a framework with no measurable periodic order or pattern to the relative arrangement of nanostructures within the framework, e.g., no regular inter-nanostructure spacings, orientation, rotation, alignment, helicity, and the like. Thus, a “disordered array of nanostructures” encompasses all three-dimensional frameworks of nanostructures in which the nanostructures exhibit some degree of intertwining or entanglement. For example, a disordered array of nanostructures can be characterized as a mesh of nanostructures, a mat of nanostructures, a net of nanostructures or the like. As used herein “average” is meant to encompass any measure of a typical value of a distribution, e.g., median, mode or mean. As used herein, a “metallized” nanostructure is a nanostructure having at least one metal-containing nanoparticle attached thereto.

As used herein, “accessible catalytic site” is meant to describe any opening formed within the macroporous network of metallized nanostructures through which reactant molecules of a specific chemical reaction can pass and thus reach the surface of the metal-containing nanoparticles deposited on the nanostructures. “Size of accessible catalytic sites” is defined as the mean of measurements of the maximum distance between adjacent nanostructures, e.g., nanosprings or nanowires, that define the opening through which the reactant molecules can pass for a sample size sufficiently large to provide a confidence interval of at least 95%. Such maximum distance between adjacent nanostructures is measured from microscopy images (e.g. transmission electron microscope images or scanning electron micrographs) of the nanostructured catalysts for an area of sufficient size to provide the confidence interval for the mean described above. In some embodiments, the size of accessible catalytic sites may range from less than about 100 nm to about several tens of microns.

“Size distribution of accessible catalytic sites” can be defined as the variance τ²=Σ(xᵢ−μ)²/N, where μ is the size of accessible catalytic sites defined above, N is the number of measurements used to generate the mean discussed above, i=1 to N, and xᵢ is the value of each individual measurement used to determine the size of accessible catalytic sites. Because the nanostructures are arranged in a disordered manner, the size distribution of accessible catalytic sites of nanostructured catalysts described herein is usually much larger than a size distribution of a nanostructured catalyst where the nanostructures are arranged in an ordered manner. A large variance in opening size of accessible catalytic sites may be advantageous. For example, a catalyst comprising a relatively wide range in opening size of accessible catalytic sites may be more effective in catalyzing a polymerization reaction than a catalyst comprising relatively uniformly-sized openings of accessible catalytic sites.

Nanostructured catalysts, methods for catalyzing reactions using the nanostructured catalysts, and examples of reactions that can be catalyzed using the nanostructured catalysts are described herein. In general, the nanostructured catalysts comprise a disordered array (mesh or mat) of nanostructures. The nanostructures can have a variety of configurations (shapes or structure types and dimensions), with non-limiting example including nanosprings, nanorods, nanowires, nanotubes, and combinations thereof. Disposed on the nanostructures within the disordered array are metal-containing (metals, metal alloys, metal oxides, metal-complexes, and the like) nanoparticles. In the nanostructured catalysts, the nanoparticles generally have a smaller cross-sectional dimension than the nanostructures to which they are attached. For example, in a disordered array, an average cross-sectional dimension of the nanoparticles may be at most about 1/100, about 1/50, about 1/20, about 1/10, about ⅕, about ¼, about ⅓, or about ½ an average cross-sectional dimension of the nanostructures in that disordered array. Examples of disordered arrays of insulating and semiconducting nanostructures that can be grown on a substrate are provided in International Patent Application No. PCT/US2006/024435, “Method for Manufacture and Coating of Nanostructured Components,” filed Jun. 23, 2006, which is incorporated herein by reference in its entirety.

Such a disordered array or mat of nanostructures may be well-suited for catalyst applications because of the potential for a very high surface area to mass ratio, which may result in increased numbers of reactive sites. The nanostructured catalysts described herein may be suitable heterogeneous or easily separable catalysts with high accessible surface ‘areas that may be specifically modified to catalyze a variety of different reactions, e.g., hydrogenation, dehydrogenation, or polymerization. In some cases, the nanostructured catalysts may be efficient at low temperatures, which may make them useful for hydrogen generation, carbon monoxide oxidation, and the reduction of organic alcohols.

In certain variations, the nanostructured catalysts described herein may be well-suited to catalyze oxidation reactions. Molecular oxygen has been shown to dissociate and fill oxygen vacancies as readily available and highly reactive atomic oxygen. See Zhou et al., J. Catal., 229 (2005), 206, which is incorporated by reference in its entirety. Gas species containing oxygen sometimes do not bind well to metal oxide surfaces without the presence of oxygen vacancies. See Maiti et al., Nano Lett. 3 (2003), 1025, which is incorporated by reference in its entirety.

The products of a catalyzed reaction or adsorbed species may desorb from a nanostructured catalyst as described herein with very little energy since the metal ions can easily change valence states, causing a change in the affinity of the products for the metal oxide surface.

The methods for catalyzing reactions described herein include providing a nanostructured catalyst that comprises metal-containing nanoparticles disposed on a substrate and/or on a disordered array of nanostructures. If present, the nanostructures may be for example nanowires, nanotubes, nanosprings, nanorods, or a combination thereof that have been grown on a substrate or otherwise provided.

Methods for generating hydrogen gas from a hydrogen-containing molecular species using a nanostructured catalyst are described herein. The hydrogen gas may be collected and stored, or the hydrogen gas may be collected and consumed as fuel, e.g., by a fuel cell or to power an engine. Thus, in some variations, the methods may be used as part of the operation of a fuel- or energy-consuming device or system, e.g., an engine or a fuel cell. Methods for storing hydrogen by using a nanostructured catalyst to react a dehydrogenated molecular species with hydrogen gas to form a hydrogen-containing molecular species that can subsequently be catalytically dehydrogenated to release hydrogen gas are also described.

The following description provides headings for convenience. However, the headings are not intended to limit or subdivide the description in any way. For example, any of the nanoparticles described below in Section I.B may be combined with any of the nanostructures described below in I.C to form nanostructured catalysts, which may in certain circumstances be used to catalyze reactions described in Sections II and III below.

### I. Examples of Nanostructured Catalysts

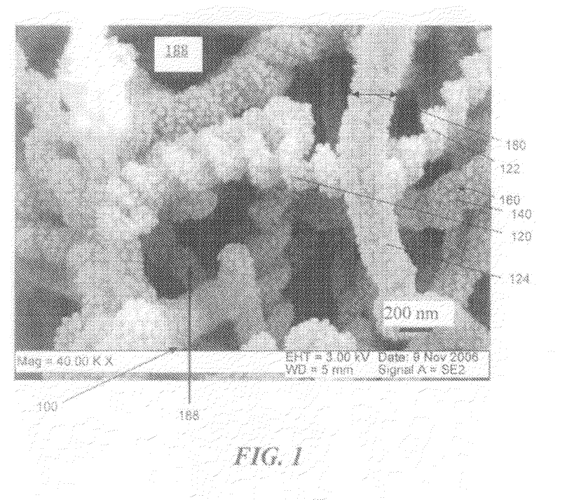

An example of a nanostructured catalyst is illustrated in FIG. 1. There, the catalyst 100 comprises nanostructures 120 (which in this particular variation are configured as a mixture of nanosprings 122 and nanowires 124). Attached to the nanostructures 120 are a plurality of metal-containing nanoparticles 140. As shown, a cross-sectional dimension 160 of the nanoparticles 140 is much less than a cross-sectional dimension 180 of the nanostructures 120. In this particular variation, the cross-sectional dimension 160 is about 1/10 the cross-sectional dimension 180, such that the nanoparticles 140 appear as fine features on the surface of the nanostructures 120. The metallized nanostructures provide a macroporous network in which essentially all of the volume within the network may be accessible as a catalytic site. That is, because of the relatively large open volumes 188 between the nanostructures 120 metallized with the nanoparticles 140, and the relative uniform presence of metal-containing nanoparticles 140 on all or most surfaces of the nanostructures, which are in turn arranged in a disordered manner, reactants are likely to be able to find an accessible catalytic site to which they can be adsorbed or bound and catalyzed by the metal-containing nanoparticles.

As is discussed in more detail herein, various properties of nanostructured catalysts, such as illustrated in FIG. 1, may be selected so as to tune the catalyst for catalyzing a particular reaction. For example, any one or any combination of the following variables may be tuned: i) the composition of the nanostructures; ii) the configurations of the nanostructures (e.g., shape or structure type, and dimensions of the structures); iii) the density of the nanostructures within the disordered array; iv) the composition of the nanoparticles; v) the size of the nanoparticles; vi) the composition of the nanoparticles; vii) the distribution of the nanoparticles on the nanostructures; viii) electronic interactions between the nanoparticles and the nanostructures; ix) the presence or absence of a semiconductor or metal oxide layer between the nanostructure and the nanoparticles; x) the size of accessible catalytic sites that are present in the macroporous network formed by the disordered array of metallized nanostructures described herein; and xi) the spatial distribution of accessible catalytic sites that are present in the macroporous network. These variables in some cases may be modified independently of each other (e.g., nanoparticle size and composition may be changed independently of the type and density of nanostructures), whereas in other cases variables may be coupled (the size of accessible catalytic sites may depend on density of nanostructures, configuration of nanostructures, nanoparticle size, and nanoparticle distribution on nanostructures). As stated above, selected combinations of these variables may be changed to modify the performance of a catalyst to suit a particular reaction. For example, in some variations, the size of accessible catalytic sites within the macroporous network may be tuned to accommodate a certain reaction, while selecting one or both of the metal in the metal-containing nanoparticle and an average nanoparticle size to catalyze that reaction.

The nanostructured catalyst may be anchored to a substrate in some variations. In other cases, the nanostructured catalyst may not be anchored to a substrate, and may be a free-standing mesh or mat.

### I.A. Examples of Nanoparticle/Nanostructure Interactions

In many catalytic systems, a molecular species may be adsorbed onto a catalyst surface. In some situations, the electronic band structure of an adsorbed molecular species may be different than that of the non-absorbed corresponding species. The electronic state of surface atoms on a nanostructure may change or be changed, e.g., to assist in the adsorption of a molecular species to the surface. The deposition of metal-containing nanoparticles (e.g., zero-dimensional particles, or particles that are sufficiently spread out so as to not form a contiguous one-dimensional, two-dimensional or three-dimensional grouping) onto a substrate or nanostructure surface may alter the electronic state of the nanoparticles and/or surface atoms on the substrate or nanostructure surface. In some instances, this may lead to novel electronic and/or catalytic properties of a nanostructured catalyst comprising the deposited nanoparticles on the substrate or nanostructure surface. For example, a nanostructured catalyst comprising metal-containing nanoparticles deposited, grown, or otherwise disposed on a semiconductor substrate, semiconductor nanostructure, or a nanostructure coated with a semiconductor coating may exhibit improved catalytic properties.

The adsorption of a molecular species (a reactant) onto the surface of a nanostructure is useful for catalyst application. A change of the electronic band structure of a material as it reaches nanoscale dimensions may be due to an effect of reduced interactions with neighboring atoms. As a result, surface atoms may have the ability to change electronic state and assist the adsorption process. Similarly, the deposition of metal-containing nanoparticles onto a semiconducting nanostructure can alter the electronic state of each of the nanoparticles and nanostructure, thereby imparting novel electronic and catalytic properties to the hierarchical nanostructured assemblies described herein.

A nanostructure may in some variations comprise a conducting channel with a cross-sectional conducting area. When a charge-withdrawing species is adsorbed onto the surface of a nanostructure, the conducting volume of the nanostructure may be effectively decreased due to a depletion region formed near and around the adsorbed species. Non-limiting examples of electronic interactions between nanoparticles and their underlying nanostructures are provided in Dobrokhotov, et al., “Principles and mechanisms of gas sensing by GaN nanowires functionalized with gold nanoparticles,” J. Appl. Phys. 99 (2006), 104302, which is incorporated by reference herein in its entirety.

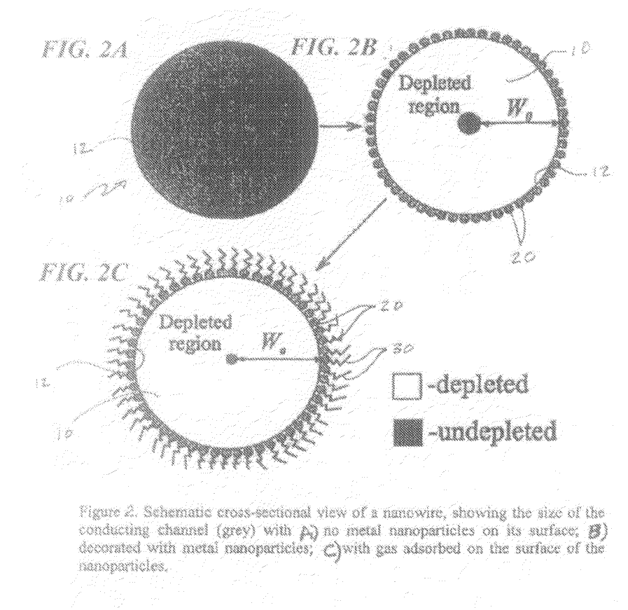

Referring now to FIGS. 2A-2C, a schematic cross-sectional view of a conducting nanowire 10 is shown. In FIG. 2A, a nanowire 10 with an uncoated surface 12 is shown as having a reasonably uniform conducting region 14 filling the cross-section of nanowire 10. In FIG. 2B, nanowire 10 is shown with metal nanoparticles 20 disposed on the surface 12. In this instance, an approximately cylindrical depleted region 16 surrounds a conducting region 14′. In this illustration, the conducting metal nanoparticles 20 have interacted with the conducting nanowire 10 such that the charge carrier density in depleted region 16 has decreased so as to effectively reduce the conductive cross-sectional area of the nanowire 10. Referring now to FIG. 2C, the adsorption of a species 30, e.g., a molecular species, onto the surface 12 decorated with nanoparticles 20 can create an even larger depleted region 18. Thus, in such a scenario where the nanoparticles interact electronically with the nanostructure, the nanostructure or the nanoparticles alone may not catalyze or effectively catalyze a reaction, but the nanoassembly comprising a particular combination of nanoparticles and nanostructure may catalyze the reaction, or improve the catalysis of the reaction. Similarly, one or more types of nanoparticles may not catalyze or effectively catalyze a reaction unless used in combination with a nanostructure made from or comprising a particular material or class of materials. Although FIGS. 2A-2C illustrate the electronic interaction between nanoparticles and a nanowire, an analogous mechanism may be operative for nanoparticles deposited on a relatively planar substrate, e.g., a semiconducting substrate or a substrate coated with a semiconducting material.

For example, in some cases, metal-containing nanoparticles may be deposited onto, grown onto, or otherwise provided on a surface of a nanowire and/or substrate that comprises a semiconductor, e.g., wide bandgap semiconductor, or a semiconductor whose bandgap is greater than about 1 eV or about 2 eV, e.g., III-V or II-VI semiconductor materials. For example, nanostructures and/or substrates comprising gallium nitride, aluminum nitride, zinc oxide, titanium dioxide, silicon carbide, carbon (e.g., diamond), copper oxide such as copper (I) oxide, copper (II) oxide or a mixture thereof, and/or cobalt oxide such as cobalt (II) oxide, cobalt (III) oxide, cobalt (IV) oxide, cobalt (II,III) oxide, cobalt (II,IV) oxide or a mixture thereof may comprise metal-containing nanoparticles on their surface, e.g., nanoparticles comprising Au, Fe, Co, Ni, Cu, Pt, Pd, Rh, Ag, and various alloys thereof. For example, nanostructures and/or substrates comprising aluminum nitride or gallium nitride may be used in combination with metal-containing nanoparticles, e.g., gold nanoparticles.

### I.B. Examples of Nanostructures

As described above, the nanostructured catalysts described here may in some variations comprise a disordered array of nanostructures, where the nanostructures may comprise a variety of structures such as nanosprings, nanowires, nanorods, nanotubes, single strand nanostructures, multistrand nanostructures, and any combination thereof. The disordered array formed by the nanostructures may be disposed on or attached to at least a portion of a substrate, which may be insulating, conductive, or semiconducting.

Nanostructures (e.g., one-dimensional nanostructures) may be well-suited for catalyst applications because of their large surface area to volume ratio, which in general results in orders of magnitude more reactive sites than thin films or bulk materials. As a point of reference, the surface are per mass of silicon nanowires with mean diameters of about 15 nm has been calculated to be about 115 m²/g, which is over about 1000% larger than that of a thick silicon film with 1 mm by 1 mm dimensions.

In general, the disordered array of nanostructures may be grown directly on a substrate, or otherwise anchored or adhered to a substrate, e.g., a binder such as a catalyst used to grow nanostructures, may be used to adhere the nanostructures to a substrate. The nanostructures used in the catalysts may comprise a glass (e.g., silica (SiO₂ or SiOₓ)), a ceramic (e.g., SiC, BN, B₄C or Si₃N₄), a ceramic oxide (e.g., Al₂O₃ or ZrO₂), a metal or semiconductor (e.g., Si, Al, C, Ge, GaN, GaAs, InP or InN).

The disordered array of nanostructures for use in the nanostructured catalysts may be grown in any suitable manner. In some variations, a disordered array of nanostructures for use in the catalysts may be grown by depositing a thin film catalyst on the substrate, heating the thin-film catalyst on the substrate together with gaseous, liquid, and/or solid nanostructure precursor material or materials, and then cooling slowly under a relatively constant flow of gas to room temperature, e.g., generally following the methods for growing nanostructures as described in International Patent Application No. PCT/US2006/024435, which has already been incorporated herein by reference in its entirety. If more than one nanostructure precursor material is used, the precursor materials may be added in a serial or parallel manner.

The concentration of precursor material(s) and/or heating time of the pretreated substrate together with the precursor material(s) may be varied to adjust properties of the resultant disordered array of nanostructures (e.g., mesh thickness and/or nanostructure density). Typical heating times are from about 15 minutes to about 60 minutes. Molecular or elemental precursors that exist as gases or low boiling liquids or solids may be used so that processing temperatures as low as about 350° C. may be used. The processing temperature may be sufficiently high for the thin film catalyst to melt, and for the molecular or elemental precursor to decompose into the desired components. These nanostructure-growing processes may allow the use of a wide variety of substrates. For example, metal, glass, semiconducting, or ceramic substrates may be used. In some variations, relatively low-melting point substrates may be used, such as aluminum, or polymeric materials that are inherently conductive (conductive polymers) or have been made conductive with conductive fillers and/or coatings (e.g., polyimides or other polymers or polymer composites having a sufficiently high T_{g} to allow relatively short excursions to about 350° C.).

The thin film catalyst may be applied to the substrate using any suitable method. For example, thin films of metal or metal alloy catalysts may be applied using plating, chemical vapor deposition, plasma enhanced chemical vapor deposition, thermal evaporation, molecular beam epitaxy, electron beam evaporation, pulsed laser deposition, sputtering, and combinations thereof. In general, the thin catalyst film is applied as a relatively uniform distribution (e.g., a contiguous or nearly contiguous uniform layer) to allow for relatively uniform growth of nanostructures. The thickness of the thin film catalyst may be varied to tune properties of the resultant mesh of nanostructures (e.g., a thickness of the mesh and/or a density of the nanostructures). In some variations, the thickness of the thin film catalyst may be from about 5 nm to about 200 nm. Non-limiting examples of materials that may be used as the thin film catalyst include Au, Ag, Fe, FeB, NiB, Fe₃B and Ni₃B. After a thin film catalyst layer has been applied to the substrate, the substrate is heated, in some cases so that the catalyst layer melts to form a liquid, and one or more nanostructure precursor materials are introduced in gaseous form so that they can diffuse into the molten catalytic material to begin catalytic growth of the nanostructures.

In some variations of these processes, a substrate pre-treated with a thin catalytic layer may be heated in a chamber at a relatively constant temperature to generate and maintain a vapor pressure of a nanostructure precursor element. In these variations, non-limiting examples of nanostructure precursor materials include SiH₄, SiH(CH₃)₃, SiCl₄, Si(CH₃)₄, GeH₄, GeCl₄, SbH₃, and AlR₃, where R may for example be a hydrocarbon.

In other variations of these processes, a substrate pre-treated with a thin catalytic layer may be heated a chamber together with a solid elemental nanostructure precursor at a relatively constant temperature that is sufficient to generate and maintain a vapor pressure of the nanostructure precursor element. In these variations, non-limiting examples of the solid elemental nanostructure precursor include C, Si, Ga, B, Al, Zr and In. In some of these variations, a second nanostructure precursor may be added into heated chamber, e.g., by introducing a flow or filling the chamber to a static pressure. Non-limiting examples of the second nanostructure precursor include CO₂, CO, NO and NO₂.

In still other variations, a pre-treated substrate may be heated in a chamber to a set temperature at least about 100° C., and a first nanostructure precursor material may be introduced into the chamber through a gas flow while the chamber is heated to the set temperature. After the chamber has reached the set temperature, the temperature may be held relatively constant at the set temperature, and a second nanostructure precursor material may be flowed into the chamber. In these variations, non-limiting examples of the first and/or second nanostructure precursor materials include SiH₄, SiH(CH₃)₃, SiCl₄, Si(CH₃)₄, GeH₄, GeCl₄, SbH₃, AlR₃ (where R is for example a hydrocarbon group), CO₂, CO, NO, NO₂, N₂, O₂, and Cl₂.

For example, to make a disordered array comprising helical silica nanostructures, a substrate capable of withstanding at least about 350° C. for about 15 to 60 minutes may be pre-treated by sputtering a thin, uniform layer of Au on the substrate (e.g., a layer about 15 nm to about 90 nm thick). To achieve the desired Au thickness, the substrate may be placed into a sputtering chamber at about 60 mTorr, and an Au deposition rate of about 10 nm/min may be used while maintaining a constant O₂ rate during deposition. The substrate that has been pre-treated with Au may be placed in a flow furnace, e.g., a standard tubular flow furnace that is operated at atmospheric pressure. A set temperature in the range of about 350° C. to about 1050° C., or even higher, may be selected depending on the substrate used. During an initial warm up period in which the furnace is heated to the set temperature, a 1 to 100 standard liters per minute (slm) flow of SiH(CH₃)₃ gas is introduced into the furnace for about 10 seconds to about 180 seconds, and then turned off. After the flow of SiH(CH₃)₃ is terminated, pure O₂ may be flowed through the furnace at a rate of about 1 to 100 slm. The furnace is then held at the set temperature for about 15 to about 60 minutes, depending on the desired properties of the disordered array of silica (SiO₂ or SiOₓ) nanostructures.

GaN nanowires may be grown, e.g., using methods described in International Patent Application No. PCT/US2006/024435, which has already been incorporated herein by reference in its entirety. GaN nanowires may be grown in a flow furnace using a ceramic boat to hold pellets of Ga. A substrate may be sputtered with a catalytic gold layer having a thickness of about 15 nm to about 90 nm. The gold-coated substrate may then be placed in a flow furnace. The furnace may be purged with nitrogen gas and heated to a temperature between 850° C. and 1050° C. After the furnace reaches the desired temperature, the nitrogen purge may be ceased and ammonia gas may be introduced into the furnace at a flow rate of 1-100 slm. The furnace may then be maintained at temperature with a flow of ammonia gas for 15-60 minutes. Alternatively, the furnace may be filled with ammonia to reach an atmospheric or higher pressure of ammonia. The furnace may be then sealed and held at temperature and pressure for 15-30 minutes. Following either approach, after the appropriate length of time the ammonia may be turned off and the system purged with nitrogen gas as it cools to ambient temperature.

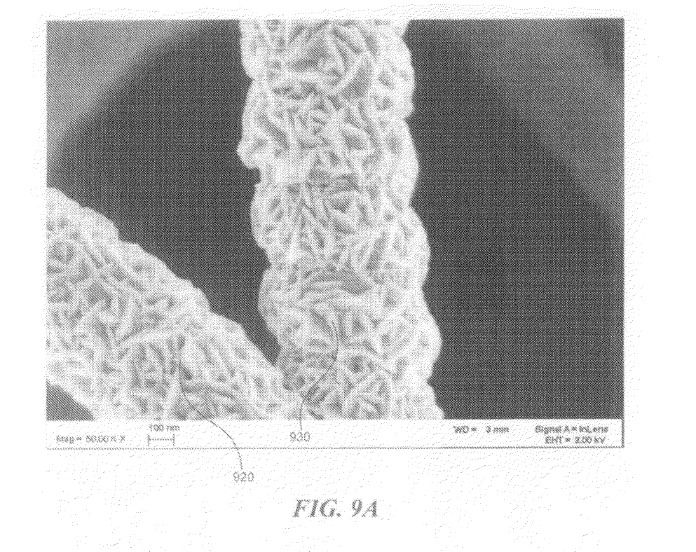

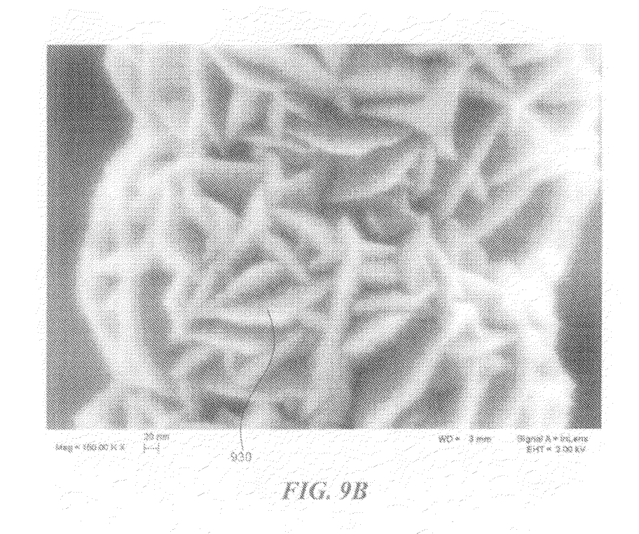

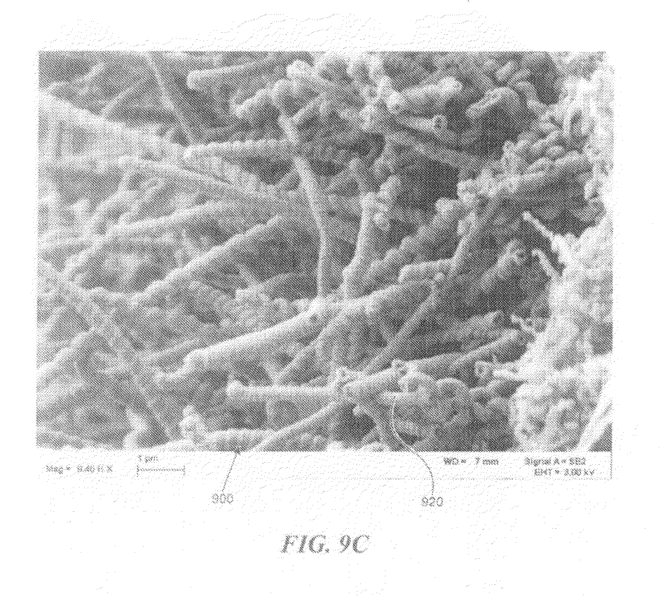

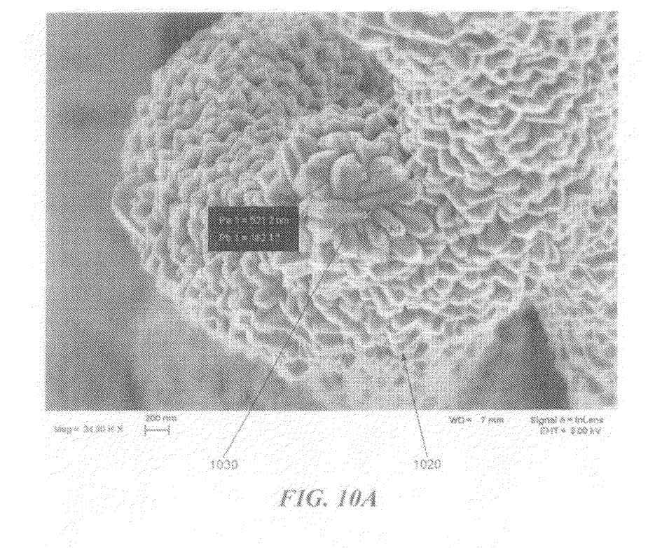

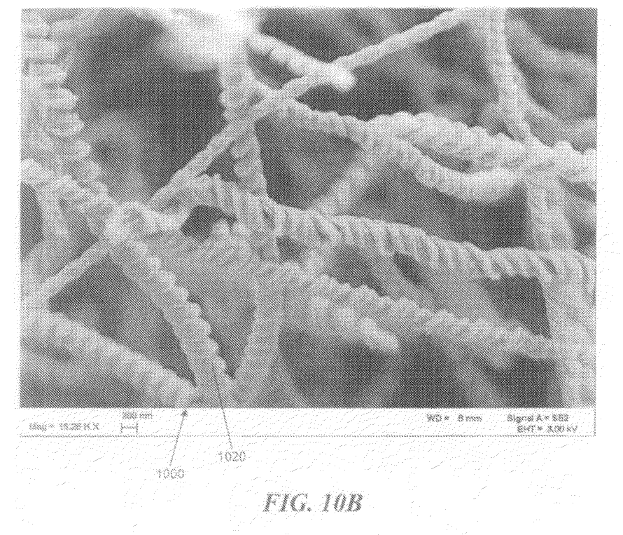

Nanostructures and/or substrates may comprise a semiconductor material to provide a conducting surface that may electronically interact with nanoparticles on the surface. Such semiconducting nanostructure and/or substrate regions may comprise one or more single crystal regions and/or one or more polycrystalline regions. Alternatively, or in addition, a semiconductor material coating may be deposited on, grown on, and/or otherwise disposed on a nanostructure surface and/or substrate surface, e.g., an insulating nanostructure such as a silica nanostructure. Such a semiconductor material layer may comprise one or more polycrystalline regions and/or one or more single crystal regions. The semiconductor material may be GaN in some variations (single crystal or polycrystalline GaN). In other variations, the semiconductor material may comprise ZnO or TiO₂, e.g., as illustrated below in FIGS. 9A-9C and 10A-10B, respectively. In yet other variations, the semiconductor material may comprise copper oxide such as copper (I) oxide, copper (II) oxide or a mixture thereof or cobalt oxide such as cobalt (II) oxide, cobalt (III) oxide, cobalt (IV) oxide cobalt (II,III) oxide, cobalt (II,IV) oxide or a mixture thereof. In certain variations, the semiconductor material may comprise Si, SiC, or AlN. The semiconductor material layer may have any suitable thickness, but in general the semiconductor layer may be characterized as a contiguous layer over the underlying nanostructure. However, the semiconductor layer may not be so thick as to unnecessarily fill in spaces between and nanostructures or nanostructure features, e.g., intracoil and/or inter-coil voids in a nanospring. For example, the semiconductor layer may be about as thick as a diameter of the underlying nanostructure, or about twice as thick as a diameter of the underlying nanostructure, or even about three or about four times as thick as a diameter of the underlying nanostructure.

In some variations, a GaN layer may be grown, deposited, and or otherwise provided on a nanostructure made from silica, e.g., a nanowire, a nanotube, or a nanospring (helical nanostructure). The silica nanostructure may be grown by any suitable technique, described herein or known in the art. The GaN layer may be formed by any suitable method, e.g., by atomic layer deposition, similar to the techniques described in E.B. Yousfi et al., “Atomic layer deposition of zinc oxide and indium sulfide layers for Cu(ln,Ga)Se₂ thin-film solar cells,” Thin Solid Films, 387 (2002) 29-32, which is hereby incorporated by reference in its entirety. A GaN layer may also be formed on silica nanostructures, e.g., nanowires or nanosprings, by using chemical vapor deposition or plasma-enhanced chemical vapor deposition. A GaN layer formed on a silica nanostructure may comprise one or more single crystal regions and/or one or more polycrystalline regions. In some variations, a GaN layer having a thickness of at least about at least about 5 nm, at least about 10 nm, at least about 15 nm, at least about 20 nm, at least about 25 nm, at least about 30 nm, or at least about 50 nm may be grown on a silica nanostructure, e.g., a silica nanostructure having a cross-sectional dimension of about 5 nm to about 500 nm, e.g., about 5 nm to about 300 nm, or about 5 nm to about 100 nm.

A range of densities of nanostructures on the substrate may be made with the methods described here. The density of nanostructures on the substrate may be varied by varying the thickness of the thin film catalyst deposited on the substrate. If the thin film catalyst layer is relatively thick (e.g., 30 nm or thicker), the nanostructures may be very densely packed with nanostructures comprising groups of intertwined and/or entangled nanostructures, e.g., nanosprings, or a combination of nanostructures. A relatively thin catalyst film (e.g., about 10 nm or thinner) may result in nanostructures that may be widely spaced apart, e.g., about 1 μm apart or even farther). For example, an areal density of nanostructures on the substrate of about 5×10⁷ nanostructures per square cm to about 1×10¹¹ nanostructures per square cm may be achieved.

The areal density of nanostructures on a substrate may be estimated using the initial thickness of the thin film catalyst layer, and the average size of the catalyst particle or droplet left at the end of each nanostructure formed. The initial thickness of the thin film catalyst layer may be determined using an atomic force microscope, by examining a border between a catalyst-coated area (e.g., a gold-coated area) and an uncoated area of the substrate. The average catalyst size may be determined from the wavelength of the catalyst plasmon (e.g., the Au plasmon) obtained from a mesh or mat formed from nanostructures, e.g., nanosprings. In some variations, multiple layers of nanostructures (e.g., nanosprings) can be formed by depositing a catalyst layer onto an existing mat or mesh, whereby nanostructures are grown on top of the existing mat or mesh by the previously described process. This catalyst may, for example, be nanoparticles (e.g., gold nanoparticles) that have been coated onto the nanostructures in the existing mat or mesh.

In some variations, each layer in a mesh or mat may have a depth of about 10 μm, and multiple layers may be built up to provide a mesh or mat that has a depth of about 20 μm, about 30 μm, about 50 μm, about 80 μm, about 100 μm, or even thicker, e.g., about 200 μm. In such a multi-layer approach, nanostructures in different layers may comprise the same or different materials, and may have the same or different shapes. For example, a mesh or mat comprising two or more layers of silica nanostructures may be fabricated, or a mesh or mat comprising one or more layers of silica nanostructure and one or more layers of nanostructure comprising a semiconductor, e.g., gallium nitride nanostructures may be fabricated.

As stated above, the nanostructures may have any suitable shape and/or dimensions. For example, the nanostructures may comprise nanowires, nanotubes, nanosprings, nanorods, nanohorns, single strand or multi-strand, or any combination thereof. The nanostructures may range from less than about a micron to about 10 microns in length, and have a cross-sectional dimension from about 5 nm to about 500 nm, e.g., about 5 nm to about 300 nm, or about 5 nm to about 100 nm. Within a mat or mesh, nanostructures having a substantial variation in cross-sectional dimensions may be present. That is, within a mat or mesh, nanostructures having a cross-sectional dimension from about 5 nm to about 100 nm, or from about 5 nm to about 200 nm, or from about 5 nm to about 300 nm, or from about 5 nm to about 400 nm, or from about 5 nm to about 500 nm, or from about 15 nm to about 500 nm, may be present. The nanostructures may have any suitable cross-sectional shape, e.g., round, oval, hexagonal, elongated (e.g., ribbon-like), and the like.

### I.C. Examples of Nanoparticles

The structure of certain nanoparticles can affect their electronic properties and their catalytic activity. The electrical properties of metal nanoparticles conducive to catalyst applications in metals can be due to quantum size effects. Metal-containing nanoparticles may serve as extremely active sites for the adsorption and dissociation of molecules due to unique electrical properties caused by quantum effects. As metal particles reach the nanometer size range, their energy bands become quantized rather than nearly continuous, as is the case in the bulk material. A shift in Fermi Energy (E_{F}) can lead to semiconductor behavior. See, e.g., Meier et al. 2002, which is incorporated by reference herein in its entirety. Semiconductor behavior can enhance the ability of the nanoparticles to adsorb and dissociate reactant species. In particular nanoparticles of metals that have either d-band vacancies or easily ionized d-bands (e.g., Au, Fe, Co, Ni, Cu, Rh, Pd and Ag) may be active in some reactions, e.g., highly active in some cases.

Gold nanoparticles that show semiconductor behavior may in some cases exhibit enhanced catalytic activity. See, e.g., Haruta, Appl. Catal. A: General, 222 (2001), 427, Haruta, The Chemical Record, 3 (2003), 75, Chung et at., Appl. Phys. Lett., 76 (2000), 2068, Hanrath et al., J. Am. Chem. Soc., 124 (2002), 1424, Guczi et al., J. Am. Chem. Soc., 125 (2003), 4332, each of which is incorporated by reference herein in its entirety. Small gold nanoparticles containing 12 or less atoms may be amorphous and may be particularly active for oxidation of CO. See Cunningham et al., J. Catal., 177 (1998), 1, which is incorporated by reference herein in its entirety.

The shape of metal nanoparticles can also influence their catalytic activity in some cases. Gold nanoparticles with 13 atoms and icosahedral symmetry may be more catalytically active than similarly sized gold nanoparticles having cuboctahedral symmetry. The icosahedral symmetry is constructed of corner atoms bonded to five other atoms, while the cuboctahedral symmetry consists of corner atoms bonded to four other atoms. The icoshedral symmetry and the cuboctahedral symmetry have different band structures. Even particles consisting of 300 gold atoms were shown to have band structures different than those exhibited by bulk gold, although the 300-atom gold particles are less catalytically active than smaller nanoparticles. See Haruta, The Chemical Record, 3 (2003), 75, which is incorporated by reference herein in its entirety. The potential energy of a gold nanoparticle is influenced by its nearest neighbors, therefore metals may have drastic changes in particle shape (geometry) and crystal structure between the bulk metal and nanoparticles. Aluminum nanoparticles exhibit a transition in geometry and crystallinity between particles containing several hundred atoms to those containing a few thousand atoms. The activity of transition metal nanoparticles has been studied and appears to optimal for some reactions for particles having sizes in a range from about 3 nm to about 6 nm. See Schogl, et al., Agnew. Chem. Int. Ed., 43 (2004), 1628, which is incorporated by reference herein in its entirety.

The catalytic activity of metal nanoparticles can also be affected by the support to which they are attached, e.g., as described above in Section I.A. For example, gold nanoparticles supported on metal oxide nanostructures have been demonstrated to act as catalysts in certain examples. See Haruta, The Chemical Record, 3 (2003), 75, Carretin el al., Agnew. Chem. Int. Ed., 43 (2004), 2538, Iizuka et al., J. Catalysis, 187 (1999), 50, Fu et al., Science, 301 (2003), 935, each of which is incorporated by reference herein in its entirety. In some cases, a phase boundary between metal nanoparticles and a ceramic support may increase catalytic activity of the metal nanoparticles. For example, for a system comprising gold nanoparticles on a TiO₂ substrate, the gold atoms bonded to Ti and O atoms on the substrate may be the most active atoms in the system. See Campbell, Science, 306 (2004), 234, which is incorporated by reference herein in its entirety.

Metal-containing (metals, metal alloys, metal oxides, metal complexes, and the like) nanoparticles particles deposited, grown and/or otherwise provided on a substrate and/or nanostructure may have any suitable composition and may be present in any suitable size range and density. In general, it may be desired to use metal-containing particles in the size range from about 2 nm to about 100 nm, or from about 2 nm to about 80 nm, or from about 2 nm to about 50 nm, or from about 2 nm to about 30 nm, or from about 2 nm to about 15 nm, or from about 5 nm to about 15 nm. In some cases, the nanoparticles individually also may have some surface structures, e.g., they may include sub-nanometer features which may contribute to their catalytic activity. In general, the covering of the metal-containing particles on the underlying nanostructure may be sufficient to provide a surface area to mass ratio of at least about 50 g/m², at least about 75 m²/g, at least about 100 m²/g, at least about 115 m²/g, at least about 125 m², at least about 150 m²/g, at least about 200 m²/g, at least about 250 m²/g, at least about 300 m²/g, at least about 400 m²/g, or at least about 500 m²/g, or even higher. For example, in some variations, a GaN nanowire may be decorated with gold nanoparticles having an average diameter of about 2 nm to about 15 nm, and may be present in such a density so as to provide a surface area to mass ration of at least about 80 m²/g, or at least about 90 m²/g, or at least about 100 m²/g, at least about 115 m²/g, or at least about120 m²/g.

In some variations, it may be desired to utilize metals that have d-band vacancies, or easily ionized d-bands for the nanoparticles in the nanostructured catalysts described here. For example, suitable metals may be Au, Fe, Ni, Cu, Rh, Pt, Pd, Fe, Ag and alloys thereof. In some cases, it may be desired to provide more than one type of nanoparticle on the nanostructures, e.g., a combination of nanoparticles made from different metals and/or metal alloys, and/or a combination of different particle size distributions of nanoparticles. In some cases, the nanoparticles themselves may comprise more than one type of metal, e.g., nanoparticles comprising an alloy may be used.

Metal-containing particles deposited, grown or otherwise provided on a substrate and/or nanostructure by any suitable technique or combination of techniques. For example, the conductive or semiconducting nanoparticles may be applied using atomic layer deposition (ALD), chemical vapor deposition (CVD), or plasma-enhanced chemical vapor deposition (PECVD). In general, the nanoparticles may have an average diameter of about 100 nm or less, about 50 nm or less, about 40 nm or less, about 30 nm or less, about 20 nm or less, or about 10 nm or less, or even smaller, about 5 nm or less, e.g., about 4 nm, about 3 nm, or about 2 nm. Further, the standard deviation of the distribution of nanoparticle diameters applied to the nanostructures may be less than about 100%, less than about 80%, less than about 50%, less than about 30%, less than about 20%, or less than about 10%. In some cases, more than one average size nanoparticle may be applied to the disordered arrays, e.g., in multiple applications. For example, a first application may apply relatively large particle sizes, e.g., about 5 to about 50 nm, and the second application may apply relatively small particles sizes, e.g., less than about 5 nm.

To achieve a desired particle size distribution and spatial distribution of metal-containing nanoparticles on a substrate and/or a disordered array of nanostructures, the nanoparticles may be deposited or grown in such a manner to control average nanoparticle size and distribution. In some variations, the nanoparticles may be grown in a parallel plate PECVD to chamber operated about 13.56 MHz. The chamber volume is about 1 cubic meter. The parallel plates are 3″ in diameter and separated by 1.5″. A nanoparticle precursor and carrier gas (e.g., argon) mixture may be introduced into the chamber from a nozzle in the center of the anode, and the sample holder may serve as a ground plate. The temperature and the pressure of the deposition process may be varied to vary the average nanoparticle size and particle size distribution. PECVD may be used to grow a variety of metal or metal alloy nanoparticles, with non-limiting examples including Au, Ag, Pt, Ni, Cu, Pd, Ru, Rh, Fe and Co. For example, dimethyl(acetylacetonate)gold(III) may be used as a precursor for gold nanoparticles, bis(cyclopentadienyl)nickel may be used as a precursor for nickel nanoparticles, and (trimethyl)methylcyclopentadienylplatinum(IV) may be used as a precursor for platinum nanoparticles. Each of these precursors is commercially available from Strem Chemicals, Newburyport, Mass.

Gold nanoparticles having small average particles sizes and narrow particle size distributions may be produced on substrates and/or nanostructures, e.g., silica or GaN substrates and/or nanostructures, using PECVD at pressures between about 17 Pa and 67 Pa, and at substrate temperatures of about 573K to about 873K. For example, gold nanoparticles having an average particle diameter of about 5 nm, with a standard deviation of 1 nm may be deposited on silica nanostructures using PECVD with a total chamber pressure of about 17 Pa, a substrate temperature of 573K, a precursor material of dimethyl(acetylacetonate)gold(III), and argon as a carrier gas. Gold nanoparticles having an average diameter of 7 nm with a standard deviation of 2 nm may be similarly produced, except with a total chamber pressure of 72 Pa and a substrate temperature of 723K. Gold nanoparticles having an average diameter of 9 nm with a standard deviation of 3 nm may be produced with a total chamber pressure of 17 Pa and a substrate temperature of 873K. Additional examples of gold nanoparticle distributions that may be formed on silica nanostructures are described in A. LaLonde et al., “Controlled Growth of Gold Nanoparticles on Silica Nanowires,” Journal of Materials Research, 20 (2005), 3021, which is hereby incorporated by reference in its entirety.

In certain variations, the nanoparticles may be distributed on the surface of a substrate and/or nanostructures such that the nanoparticles are generally isolated from each other. For example, there may be physical contact between at most about 10%, at most about 20%, at most about 30%, at most about 40%, or at most about 50% of the particles. If there is physical contact between particles, the contact may be such that adjacent particles abut each other, but a boundary or relatively clear demarcation exists between two abutting particles. The coverage of the underlying nanowire may in general not be complete. In some variations, the coverage of the Pt nanoparticles on the SiO₂ nanowire surface may be about 50% to about 90%, e.g., about 50%, about 60%, about 70%, about 80%, or about 90%. In other variations, the coverage of the nanoparticles on the nanowire surface may be at or close to 100%. In some instances, a boundary separating adjacent or abutting nanoparticles may comprise a disruption between lattice planes of the nanoparticles, e.g., lattice planes across the boundary may not be continuous, there may be a rotation or twist of the lattice planes across the boundary, and/or the lattice planes may be separated by a facet plane at the boundary.

In some variations, the metal-containing nanoparticles on the nanostructures may be metal oxides, e.g., zinc oxide, titanium dioxide, or indium tin oxide. In some variations, nanoparticles of metal oxides may be applied to nanostructures using atomic layer deposition (ALD). In these variations, the nanoparticle size and distribution may be controlled by varying the nanoparticle precursor material pressure, purge time, and number of deposition cycles. For example, nanostructures may be metallized with a contiguous uniform coating of zinc oxide nanoparticles, titanium dioxide nanoparticles, indium tin oxide nanoparticles having an average dimension of about 100 nm or smaller, or about 50 nm or smaller. An example of a catalyst comprising SiO₂ nanostructures with ZnO nanoparticles is illustrated in FIGS. 9A-9C, and an example of SiO₂ nanostructures with TiO₂ nanoparticles is illustrated in FIGS. 10A-10B. For depositing metal oxides, any suitable atomic layer deposition conditions known in the art may be used. For example, zinc oxide nanoparticles may be deposited using the procedures disclosed in E. B. Yousfi et al., “Atomic layer deposition of zinc oxide and indium sulfide layers for Cu(In,Ga)Se₂ thin-film solar cells,” Thin Solid Films, 387 (2002) 29, which is hereby incorporated by reference in its entirety.

In some cases, metal-containing nanoparticles may be deposited, grown or otherwise provided on a semiconductor layer that has been applied to a nanostructure, as described above in Section I.B. For example, a semiconductor layer (e.g., GaN, ZnO, TiO₂, copper oxide such as copper (I) oxide, copper (II) oxide or a mixture thereof or cobalt oxide such as cobalt (II) oxide, cobalt (III) oxide, cobalt (IV) oxide, cobalt (II,III) oxide, cobalt (II,IV) oxide or a mixture thereof) may be applied to a silica nanostructure, and metal nanoparticles (e.g., gold nanoparticles) may, in turn, be deposited onto the semiconductor layer.

### II. Examples of Modifiying Nanostructured Catalysts for Catalyzing Specific Reactions

In a heterogeneously catalyzed chemical reaction, the catalyst provides catalytic sites on its surface for the reactants to diffuse to and to adsorb onto. After the reaction, the products desorb from these sites and then diffuse away. The number of catalytic sites on the surface of a catalyst and the accessibility of these sites are thus important factors in affecting reaction rates. Nanostructured catalysts with high accessible surface area as described herein thus provide a good platform for heterogeneous catalysis chemical reactions. One specific advantage of the nanostructured catalysts described herein is that the catalytic activity and/or selectivity of the catalysts can be modified or tuned to catalyze specific chemical reactions by selecting: 1) the size of the accessible catalytic sites in the macroporous network; 2) at least the identity of one of the metals contained in the nanoparticles and the average cross-sectional dimension of the nanoparticles; and 3) the compositions of the nanoparticles and nanostructures where there is an electronic interaction between the nanostructures and the nanoparticles.

The hierarchical structure of the nanostructured catalysts described herein provide many levers (which may in many cases be independently varied) with which to adjust reactivity and/or selectivity of the catalysts, for example: i) the composition of the nanostructures; ii) the configurations of the nanostructures (e.g., shape or structure type, and dimensions of the structures); iii) the density of the nanostructures within the disordered array; iv) the composition of the nanoparticles; v) the size of the nanoparticles; vi) the composition of the nanoparticles; vii) the distribution of the nanoparticles on the nanostructures; viii) electronic interactions between the nanoparticles and the nanostructures; ix) the presence or absence of a semiconducting or metal oxide layer between the nanostructure and the nanoparticles; x) the size of accessible catalytic sites that are present in the macroporous network formed by the disordered array of metallized nanostructures described herein; and xi) the spatial distribution of accessible catalytic sites that are present in the macroporous network. Any one of or any combination of these variables may be changed to adapt the nanostructured catalysts for a specific reaction, so that they are versatile and broadly applicable.

In one variation, the catalytic activity of the nanostructured catalyst is modified or tuned by selecting the size of the accessible catalytic sites in the macroporous network. Accessible catalytic sites with a certain size may allow some molecules of certain size, shape and/or physical state to enter under the reaction conditions while excluding others. For example, if the reactant of a specific chemical reaction is in its liquid phase under the reaction conditions, the rate-limiting step of this catalyzed chemical reaction can be the diffusion of the reactant molecules into the accessible catalytic sites. In such case, a nanostructured catalyst with large size of accessible catalytic sites might be preferred over one with relative small size of accessible catalytic sites. However, the total number of accessible catalytic sites is typically diminished when a large size of accessible catalytic sites is selected to enhance the diffusion process of reactant molecules (all other factors being equal) because of fewer nanostructures and nanoparticles being present in the catalyst. As a result, the size of accessible catalytic sites may be selected to provide the desired level of catalytic activity. Further, the size of accessible catalytic sites may be selected to provide both the desired level of activity and selectivity for e.g. a mixture of reactants that the catalyst will contact. The size of accessible catalytic sites may be selected to admit smaller molecules while excluding most large molecules from contacting nanoparticles beneath the outer surface of and within the disordered array of nanostructures. One may therefore select the size of accessible catalytic sites to allow a desired rate of diffusion, activity, and selectivity for a particular reactant to contact the catalyst.

In some embodiments, the size of accessible catalytic sites may be tuned by adjusting the configurations of the nanostructures in the disordered array. In the disordered array, the nanostructures may be selected from the group consisting of nanowires, nanotubes, nanorods, nanosprings, and combinations thereof. The nanostructures in a disordered array may have similar configurations (e.g., essentially all nanosprings) or different structures (e.g., a mixture of nanowires and nanosprings).

In some other variations, the size of accessible catalytic sites may be modified or tuned by choosing different cross-sectional shapes of the nanostructures in the disordered array. The cross-sectional shape can be round, oval, hexagonal or elongated (e.g., ribbon-like), and the like.

Alternatively to or in addition to varying the nanostructure configurations and/or cross-sectional shape of nanostructures in a disordered array, the size of accessible catalytic sites may be modified or tuned by selecting a density, and/or thickness, and/or length of nanostructures in the disordered array. As described above, in some variations, the nanostructures may be grown by depositing a thin film catalyst on the substrate, heating the thin-film catalyst on the substrate together with gaseous, liquid, and/or solid nanostructure precursor material(s), and then cooling slowly under a relatively constant flow of gas to room temperature. In some variations, the density, thickness and length of nanostructures can be adjusted by varying precursor material(s), and/or adjusting the concentration of precursor material(s), and/or heating time of the pretreated substrate, and/or the thickness of the thin film catalyst. Further, the nanostructures in a disordered array may have similar dimensions (e.g., having similar cross-sectional dimensions or lengths) or different dimensions (e.g., a mixture of relatively thick nanostructures with relatively thin nanowires or a mixture of relatively long nanostructures with relatively short nanostructures).

In some other variations, the catalytic activity and/or selectivity of the catalysts is modified or tuned by selecting at least one of metal-containing nanoparticles and the average cross-sectional dimension of the nanoparticles. In some variations, the metal-containing nanoparticles may be Au, Fe, Ni, Cu, Rh, Ru, Pt, Pd, Fe, Ag and alloys and combinations thereof. In some variations, the average cross-sectional dimension of the nanoparticles may be in the size range from about 2 nm to about 100 nm, or from about 2 nm to about 80 nm, or from about 2 nm to about 50 nm, or from about 2 nm to about 30 nm, or from about 2 nm to about 15 nm, or from about 5 nm to about 15 nm.

The nanostructured catalysts described herein can be engineered to catalyze a range of different chemical reactions depending on the specific material combination of the nanostructure support and the metal-containing nanoparticles and the specific size of accessible catalytic sites. For example, for certain reactions with larger reactants molecules, a catalysis device comprising a macroporous network comprising relatively fewer accessible catalytic sites but having relatively large size of accessible catalytic sites may be preferred over a catalysis device comprising a macroporous network comprising relatively more catalytic sites having relatively small size of accessible catalytic sites.

In one example, a nanostructure comprising SiO₂ nanostructures with TiO₂ nanoparticles (as illustrated in FIGS. 10A-10B and described below) may be used to catalyze polymerization of ethylene or propylene, e.g., using conditions similar to those described in de Souza et al., Applied Catalysis A: General, 323 (2007) 234, which is incorporated by reference herein in its entirety. Mats of silica nanosprings are an ideal substrate for the TiO₂ nanoparticles as the mats provide very high accessible surface area, which is essential for efficient reaction conversion. Silica nanostructures also provide high thermal stability and chemical durability.

In another example, a nanostructured catalyst comprising precious metal nanoparticles deposited on nanostructured substrate may be used to catalyze oxidative hydrogenation, e.g., of alkynes. For example, a nanostructure comprising SiO₂ nanostructures with Pd nanoparticles may be used to catalyze hydrogenation of alkynes, e.g., using conditions similar to those described in Teschner et al., Science, 320 (2008) 86, which is incorporated by reference herein in its entirety. The average size of the precious metal nanoparticles has an important effect on the efficiency of the hydrogenation conversion. Once the average size of the precious metal nanoparticles is too big, the efficiency of the hydrogenation conversion drops significantly. The composition of the nanostructures can be tuned to control the average size of is the precious metal nanoparticles, which in turn, control the efficiency of the hydrogenation conversion. For example, the average size of the precious metal nanoparticles deposited on silica nanosprings is smaller than that of the precious metal nanoparticles deposited on GaN under the same growth conditions. As a result, a nanostructured catalyst comprising of Pd nanoparticles deposited on silica nanosprings provides a higher efficiency of hydrogenation of alkynes than a nanostructured catalyst comprising Pd nanoparticles deposited on GaN nanosprings under the same reaction conditions.

The nanostructured catalysts described herein may be used for the catalytic reaction of various liquids and/or gases to produce carbon dioxide and hydrogen and/or a reduced organic molecules (e.g., a water gas shift reaction). For example mats of GaN nanowires with gold nanoparticles may be used to produce hydrogen from a mixture containing carbon monoxide and water at room temperature. Both carbon monoxide and water are Lewis bases, and thus have the ability to donate electron density (via surface association) to metal-containing nanoparticles. Carbon monoxide can also accept electron density from the metal particle via a backbonding interaction. In a backbonding interaction, electron density is transferred into the antibonding orbitals of the carbon monoxide π-bond, weakening the C—O bond and labalizing the heterodiatomic molecule. Under these conditions, and in the presence of a second Lewis base, an atom transfer reaction can be catalyzed. Thus, atom transfer reactivity, wherein an oxygen atom is transferred to a carbon monoxide molecule to yield carbon dioxide and a reduced oxygen donor molecule is catalyzed at the interface of a nanostructure comprising a wide bandgap semiconductor and a metal-containing (e.g., metal or metal alloy) nanoparticle.

The elements for reactivity comprise an oxygen acceptor that can be activated upon interaction with a metal surface (e.g., carbon monoxide, ketones, carbon sulfide, carbon nitride or similar molecules containing π-bonds) and an oxygen donor capable of coordinating a metal surface (e.g., H₂O, CH₃OH and other organic alcohols). The particular products will be determined by the identity of the particular donor and acceptor used, with the common element being an oxygen atom transfer from the donor to the acceptor. In one particular embodiment, the oxygen atom from water is transferred to the carbon monoxide yielding carbon dioxide and hydrogen.

### III. Molecular Species for Storing Hydrogen

Any suitable hydrogen-containing molecular species may be adsorbed onto the surface of the nanostructured catalyst decorated with metal-containing nanoparticles as described above. Such a nanostructured catalyst may catalyze the release of hydrogen from the molecular species so that the hydrogen may be recovered and used as fuel. In many cases, it is desired that the hydrogen-containing molecular species be liquid, or be capable of carried in or formulated with a liquid, for ease of handling, delivery and storage. For example, some variations of the hydrogen-containing molecular species may be capable of being carried in an alcohol formulation, e.g., an ethanol formulation.

Some examples of molecular species that can be used to store hydrogen are described in U.S. Pat. No. 7,186,396 (Ratner et al., issued Mar. 6, 2007), U.S. Patent Publication No. 2007/0003476 (Ratner et al., published Jan. 4, 2007), each of which is hereby incorporated by reference in its entirety. Other examples of molecular species that may be used to store hydrogen are described in Kariya et al., “Efficient evolution of hydrogen from liquid cycloalkanes over Pt-containing catalysts supported on active carbons under ‘wet-dry multiphase conditions,’” Applied Catalysis A 233 (2002) 91-102, which is also hereby incorporated by reference in its entirety.

In some variations, the nanostructured catalysts as described herein may be used in combination with a molecular species having the formula R—XH to generate and store hydrogen. X is in general a reactive group that is capable of binding to the nanostructured catalyst, e.g., to the metal or metal alloy nanoparticles disposed on the nanostructure mesh, and releasing hydrogen. For example, X may be a heteroatom, e.g., sulfur, oxygen or selenium. In some variations, X may be sulfur, and R—SH may be chemisorbed onto the surface of metal portions of the nanostructured catalyst. The R—XH molecular species may interact with the nanostructured catalyst in any suitable manner, e.g., one or more monolayer regions of R—XH molecules may be formed on the surface of metal portions of the nanostructured catalyst, e.g., on the metal or metal alloy nanoparticles that are disposed on the nanostructures and/or the substrate. In some instances, the R—XH molecules may form an aligned or self-assembled monolayer on the surface of metal portions of the nanostructured catalyst. R may be any suitable organic moiety, Nonlimiting examples include an alkyl, e.g., a lower alkyl, a cycloalkyl, an arylalkyl, a to heteroalkyl, an alkenyl, a substituted alkenyl, an alkenylaryl, an alkynyl, an alkynylaryl, an aryl, a heteroaryl, an alkylaryl, alkoxy, heterocyclic, arylene, oxylarylene, and combinations thereof. In some cases, R groups may contain more than one heteroatom.

The R—XH molecules may be combined with the nanostructured catalysts as described herein to generate hydrogen. The R—XH molecules may interact with metal or metal alloy nanoparticles on the nanostructures to generate hydrogen gas and to form a species bound with the catalyst, e.g., the metal or metal alloy nanoparticles, and/or a spent compound. For example, the following reaction may occur:

2(R—XH)+Metal→Metal(-X—R)₂+H₂ (gas), where the metal may be any suitable metal.

For example, if X is sulfur, a nanostructured catalyst comprising gold nanoparticles on a disordered array of nanostructures, e.g., nanostructures comprising a semiconductor which may be a wide bandgap semiconductor, such as GaN, may be used.

Non-limiting examples of alkyl groups that may be used in the R—XH molecules include straight or branched saturated hydrocarbons having from 1 to 6 (a lower alkyl), or from 1 to 12, or from 1 to 20, carbon atoms. For example, R groups may comprise methyl, ethyl, n-propyl, isopropyl, n-butyl, t-butyl, isobutyl, pentyl, cyclopentyl, hexyl, cyclohexyl, heptyl, cylohexyl, octyl, cyclooctyl, and the like. Substituted alkyl groups may be used, including hydroxyl-, alkoxyl-, mercapto-, cycloalkyl-, heterocyclic-, aryl-, heteroaryl-, arloxyl-, halogen-, cyano-, nitro-, amino-, amido-, an aldehyde group, acyl-, oxylacyl-, carboxyl-, sulfonyl-, sulfonamide- and sulfuryl-substituted alkyl groups. Nonlimiting examples of alkenyl groups that may be used in the R—XH molecules include straight or branched unsaturated hydrocarbons comprising one or more C—C bonds, e.g., C2, C3, C4, C5, C6, C7, C8, C9, C10, C11, C12, C13, C14, C15, C16, C17, C18, C19, C20, or even longer hydrocarbon chains. Alkenyl groups may be substituted, e.g., with any of the groups described in connection with substituted alkyl groups above. Nonlimiting examples of alkynyl groups that may be used in the R—XH molecules include straight or branched unsaturated hydrocarbons comprising one or more C—C triple bonds, e.g., C2, C3, C4, C5, C6, C7, C8, C9, C10, C 11, C12, C13, C14, C15, C16, C17, C18, C19, C20, or even longer hydrocarbon chains. Alkynyl groups may also be substituted, e.g., with any of the groups described above in connection with substituted alkyls.

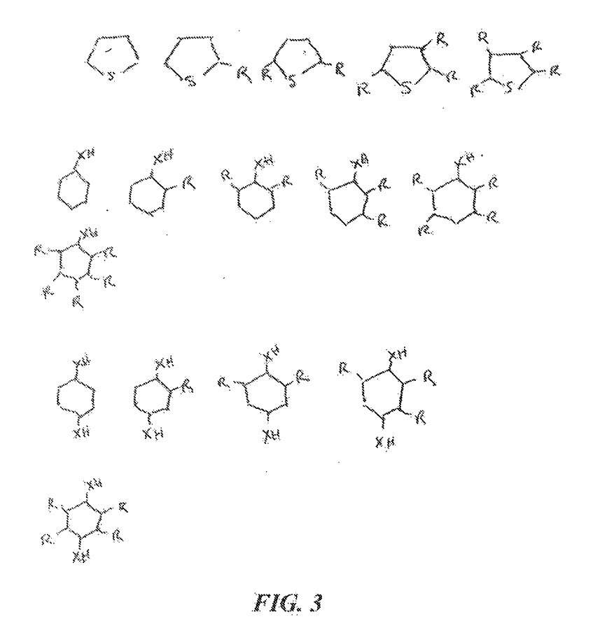

In some variations, the R—XH molecule may comprise an organothiol, e.g., CH₃SH, CH₃(CH₂)ₙSH, where n=1, 2, 3, 4, or 5. In some cases, R—XH molecules comprising multiple thiol groups may be used, e.g., two or three thiol groups. For example, dithiols may be used such as HS—(CH2)ₙ-SH (n=2, 3, 4, or 5), or HS—CH₂—CH(OH)—CH(OH)—CH₂—SH (dithiothreitol). FIG. 3 illustrates non-limiting examples R—XH molecular species that may be used with the nanostructured catalysts described herein.

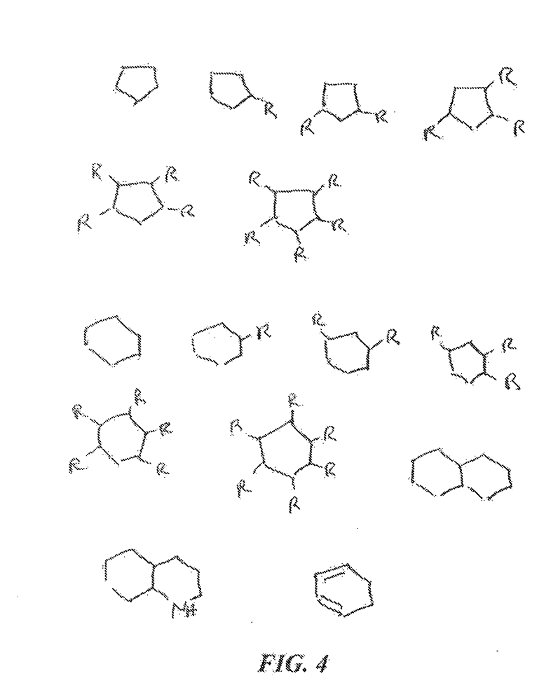

Other molecular species may be used in combination with the catalysts described herein to store and release hydrogen. In some cases, hydrocarbon molecules that do not include a heteroatom may be dehydrogenated using the nanostructured catalysts described herein. For example, any of the nanostructured catalysts described in connection with R—SH molecules may also be used in combination with any of the molecules illustrated in FIG. 4. For example, cyclohexane may be reversibly dehydrogenated in the presence of a nanostructured catalyst described here to generate hydrogen gas and benzene, methylcyclohexane may be reversibly dehydrogenated in the presence of a nanostructured catalyst described here to generate hydrogen gas and toluene, and decalin (cis-decalin or trans-decalin) may be dehydrogenated in the presence of a nanostructured catalyst described here to generate hydrogen gas and naphthalene.

Of course, the nanostructured catalyst may be tuned according to the molecular species with which it is reacting to generate hydrogen. Any suitable parameter, e.g., nanostructure size, density and/or composition, and/or metal nanoparticle size, density and/or composition may be tuned to increase catalytic efficiency and/or to adapt the system for use under certain conditions, e.g., temperature, atmosphere, and/or pressure. Some variations of nanostructured catalysts may incorporate more than one type of metal nanoparticle. If more than one type of metal is used, a mixture of nanoparticles of each type of metals, or metal alloys, or both mixtures of nanoparticles of various types of metals and metal alloys may be used. For example, the dehydrogenation of cycloalkanes, e.g., cyclohexane, methylcyclohexane, and decalin (cis-decalin or trans-decalin) may be dehydrogenated using the nanostructured catalysts described here, e.g., nanostructured catalysts comprising Pt, Pd, Rh and/or Ru nanoparticles, e.g., metal alloy nanoparticles such as Pt—Pd nanoparticles (about 1:1 ratio, or about 2:1 ratio), or Pt—Rh nanoparticles (about 1:1 ratio, or about 2.25:1 ratio). Other suitable metal and metal sources are described in Kariya et al. (2002), which has already been incorporated by reference in its entirety.

Many combinations of nanostructured catalysts and hydrogen-complexing molecular species may be used. Tables I and II show various combinations of nanostructured catalysts that may be used with each molecular species of hydrogen-storing compound, e.g., R—XH molecules or cycloalkanes. In each of the variations shown in Tables I and II, if present, the nanostructures in the nanostructured mesh or mat may have a cross-sectional dimension of about 5 nm to about 500 nm, e.g., about 5 nm to about 300 nm, or about 5 nm to about 200 nm, or about 5 nm to about 100 nm, or about 15 nm to about 100 nm. In Tables I and II, a nanostructured catalyst that may be used in combination with any of the hydrogen-containing molecular species compound may include any feature in column 1A in combination with any feature in column 2A, column 2B, column 3A, column 3B, column 3C, column 3D, column 3E, or column 3F. Further, a nanostructured catalyst may include any feature in column 1B in combination with any feature in column 2A, column 2B, column 3A, column 3B, column 3C, column 3D, column 3E, or column 3F, a nanostructured catalyst may include any feature in column 2A in combination with any feature in column 1A, column 1B, column 3A, column 3B, column 3C, column 3D, column 3E, or column 3F, a nanostructured catalyst may include any feature in column 2B in combination with any feature in column 1A, column 1B, column 3A, column 3B, column 3C, column 3D, column 3E, or column 3F, a nanostructured catalyst may include any feature in column 3A in combination with any feature in column 1A, column 1B, column 2A, or column 2B, a nanostructured catalyst may include any feature in column 3B in combination with any feature in column 1A, column 1B, column 2A, or column 2B, a nanostructured catalyst may include any feature in column 3C in combination with any feature in column 1A, column 1B, column 2A, or column 2B, a nanostructured catalyst may include any feature in column 3D in combination with any feature in column 1A, column 1B, column 2A, or column 2B, a nanostructured catalyst may include any feature in column 3E in combination with any feature in column 1A, column 1B, column 2A, or column 2B, and a nanostructured catalyst may include any feature in column 3F in combination with any feature in column 1A, column 1B, column 2A, or column 2B. The nanostructures used in the catalysts may comprise an insulator, e.g., SiO₂, and/or a semiconductor, e.g., GaN nanowires or a GaN coating on SiO₂ nanostructures, and the nanoparticles may be e.g., Au in each of the combinations disclosed in Table I. The nanostructures used in the catalysts may comprise an insulator, e.g., SiO₂, and/or a semiconductor, e.g., GaN nanowires or a GaN coating on SiO₂ nanostructures, and the nanoparticles may be e.g., Pt in each of the combinations disclosed in Table 11. Thus, for example, any R—XH molecule, e.g., R—SH, may be used with a nanostructured catalyst comprising GaN decorated with gold nanoparticles having a dimension of about 2 nm to about 15 nm and a surface/mass ratio of at least about 50 m²/g, or at least about 100 m²/g. In some variations, 1,4-cyclohexanedithiol may be used in combination with a nanostructured catalyst comprising a disordered array of GaN nanowires having a dimension of about 10 nm to about 500 nm decorated with gold nanoparticle having a dimension of about 2 nm to about 15 nm, and a surface/mass ratio of about 60 m²/g, about 70 m²/g, about 80 m²/g, about 90 m²/g, about 100 m²/g, about 110 m²/g, about 120 m²/g, about 130 m²/g, about 140 m²/g, or about 150 m²/g.

TABLE I

| | | Nanostructured catalyst comprising a substrate and/or nanostructures comprising | | | | | | | | | |
|---|---|---|---|---|---|---|---|---|---|---|---|
| | | an insulator (e.g., SiO₂) and/or a semiconductor (e.g., Si, GaN, AlN, TiO₂, ZnO, | | | | | | | | | |
| | | SiC, copper oxide, cobalt oxide) | | | | | | | | | |
| | | 1A | 1B | | | | | | | | |
| | | Metal- | Metal- | | | | | | | | |
| | | containing | containing | | | 3A | 3B | 3C | 3D | 3E | 3F |
| | | particle | particle | 2A | 2B | Metal-containing particles comprising: | | | | | |
| | | size | size | Surface | Surface | | | | | | Metal |
| Hydrogen- | | about | about | area/mass | area/mass | | | | | | alloys, |
| containing | | 2 nm to | 2 nm to | ratio of at | ratio of at | | | | | Pd, | e.g., |
| molecular | | about | about | least about | least about | | | | | Ru, or | Pt—Pd, |
| species: | | 100 nm | 15 nm | 50 m²/g | 100 m²/g | Au | Ag | Pt | Cu | Rh | or Pd—Rh |
| R—XH, where | | X | X | X | X | X | X | X | X | X | X |
| X = S, Se, or O | | | | | | | | | | | |
| R | Alkyl, e.g., | X | X | X | X | X | X | X | X | X | X |
| | straight or | | | | | | | | | | |
| | branched | | | | | | | | | | |
| | C1-C20, | | | | | | | | | | |
| | e.g., methyl, | | | | | | | | | | |
| | propyl, | | | | | | | | | | |
| | butyl, | | | | | | | | | | |
| | pentyl, | | | | | | | | | | |
| | hexyl, | | | | | | | | | | |
| | heptyl, or | | | | | | | | | | |
| | octyl | | | | | | | | | | |
| | Heteroalkyl, | X | X | X | X | X | X | X | X | X | X |
| | e.g., alkyl | | | | | | | | | | |
| | group with | | | | | | | | | | |
| | one or more | | | | | | | | | | |
| | N, S, O, or | | | | | | | | | | |
| | Se | | | | | | | | | | |
| | heteroatoms | | | | | | | | | | |
| | Alkenyl, | X | X | X | X | X | X | X | X | X | X |
| | e.g., straight | | | | | | | | | | |
| | or branched | | | | | | | | | | |
| | C2-C20, | | | | | | | | | | |
| | e.g., C2-C12 | | | | | | | | | | |
| | Substituted | X | X | X | X | X | X | X | X | X | X |
| | alkenyl | | | | | | | | | | |
| | Alkynyl, | X | X | X | X | X | X | X | X | X | X |
| | e.g., straight | | | | | | | | | | |
| | or branched | | | | | | | | | | |
| | C2-C20, | | | | | | | | | | |
| | e.g., C2-12 | | | | | | | | | | |
| | Aryl, e.g., | X | X | X | X | X | X | X | X | X | X |
| | aromatic | | | | | | | | | | |
| | rings | | | | | | | | | | |
| | including | | | | | | | | | | |
| | about 6 to | | | | | | | | | | |
| | about 14 C | | | | | | | | | | |
| | atoms | | | | | | | | | | |
| | Heteroaryl, | X | X | X | X | X | X | X | X | X | X |
| | e.g., | | | | | | | | | | |
| | aromatic | | | | | | | | | | |
| | rings | | | | | | | | | | |
| | including | | | | | | | | | | |
| | one or more | | | | | | | | | | |
| | N, O, S, or | | | | | | | | | | |
| | Se | | | | | | | | | | |
| | heteroatoms | | | | | | | | | | |
| | and about 2 | | | | | | | | | | |
| | to about 14 | | | | | | | | | | |
| | C atoms | | | | | | | | | | |
| | Alkoxy, | X | X | X | X | X | X | X | X | X | X |
| | e.g., CH₃O—, | | | | | | | | | | |
| | C₂H₅O—, | | | | | | | | | | |
| | C₃H₇O—, | | | | | | | | | | |
| | C₄H₉O—, | | | | | | | | | | |
| | C₅H₁₁O—, or | | | | | | | | | | |
| | C₆H₁₃O— | | | | | | | | | | |
| | Cycloalkyl, | X | X | X | X | X | X | X | X | X | X |
| | e.g., | | | | | | | | | | |
| | cyclopropyl, | | | | | | | | | | |
| | cylcobutyl, | | | | | | | | | | |
| | cyclopentyl, | | | | | | | | | | |
| | cyclohexyl, | | | | | | | | | | |
| | cycloheptyl, | | | | | | | | | | |
| | or | | | | | | | | | | |
| | cyclooctyl | | | | | | | | | | |
| | Heterocyclic, | X | X | X | X | X | X | X | X | X | X |
| | e.g., cyclic | | | | | | | | | | |
| | groups with | | | | | | | | | | |
| | 1 or more N, | | | | | | | | | | |
| | O, S, or Se | | | | | | | | | | |
| | atoms; e.g., | | | | | | | | | | |
| | 3, 4, 5, 6, 7, | | | | | | | | | | |
| | 8, 9, 10, 11, | | | | | | | | | | |
| | 12, 13 or 14 | | | | | | | | | | |
| | membered | | | | | | | | | | |
| | rings | | | | | | | | | | |
| | including a | | | | | | | | | | |
| | heteroatom. | | | | | | | | | | |
| | Alkyl- | X | X | X | X | X | X | X | X | X | X |
| | substituted | | | | | | | | | | |
| | aryl groups | | | | | | | | | | |
| | Aryl- | X | X | X | X | X | X | X | X | X | X |
| | substituted | | | | | | | | | | |
| | alkyl groups | | | | | | | | | | |
| | Aryl- | X | X | X | X | X | X | X | X | X | X |
| | substituted | | | | | | | | | | |
| | alkenyl | | | | | | | | | | |
| | groups | | | | | | | | | | |
| | Aryl- | X | X | X | X | X | X | X | X | X | X |
| | substituted | | | | | | | | | | |
| | alkynyl | | | | | | | | | | |
| | groups | | | | | | | | | | |
| | Arylenes, | X | X | X | X | X | X | X | X | X | X |
| | including | | | | | | | | | | |
| | divalent | | | | | | | | | | |
| | aromatic | | | | | | | | | | |
| | groups | | | | | | | | | | |
| | including | | | | | | | | | | |
| | about 6 up | | | | | | | | | | |
| | to about 14 | | | | | | | | | | |
| | C atoms | | | | | | | | | | |
| | Oxyarylene | X | X | X | X | X | X | X | X | X | X |
| | Substituted | X | X | X | X | X | X | X | X | X | X |
| | arylenes | | | | | | | | | | |
| | Butanethiol | X | X | X | X | X | X | X | X | X | X |
| | Pentanethiol | X | X | X | X | X | X | X | X | X | X |
| | Hexanethiol | X | X | X | X | X | X | X | X | X | X |
| | Cyclohexan | X | X | X | X | X | X | X | X | X | X |
| | ethiol | | | | | | | | | | |
| | 1,4- | X | X | X | X | X | X | X | X | X | X |
| | Cyclohexan | | | | | | | | | | |
| | edithiol | | | | | | | | | | |
| | Methyl | X | X | X | X | X | X | X | X | X | X |
| | mercaptan | | | | | | | | | | |
| | CH₃SH | | | | | | | | | | |
| | Thiobenzene | X | X | X | X | X | X | X | X | X | X |
| | Thiophenol | X | X | X | X | X | X | X | X | X | X |
| | CH₃(CH2)₂SH | X | X | X | X | X | X | X | X | X | X |
| | CH₃(CH2)₃SH | X | X | X | X | X | X | X | X | X | X |
| | CH₃(CH2)₄SH | X | X | X | X | X | X | X | X | X | X |
| | CH₃(CH2)₅SH | X | X | X | X | X | X | X | X | X | X |
| | Any | X | X | X | X | X | X | X | X | X | X |
| | compound | | | | | | | | | | |
| | shown in | | | | | | | | | | |
| | FIG. 3 | | | | | | | | | | |

| | | | | | | | | | | |
|---|---|---|---|---|---|---|---|---|---|---|
| Any | X | X | X | X | X | X | X | X | X | X |
| compound | | | | | | | | | | |
| shown in FIG. 4 | | | | | | | | | | |
| Cycloalkanes | X | X | X | X | X | X | X | X | X | X |
| Cyclohexane | X | X | X | X | X | X | X | X | X | X |
| Methylcyclohexane | X | X | X | X | X | X | X | X | X | X |
| Decalin (cis- | X | X | X | X | X | X | X | X | X | X |
| or trans- | | | | | | | | | | |
| decalin) | | | | | | | | | | |

TABLE II

| | Nanostructured catalyst comprising a substrate and/or nanostructures comprising an insulator (e.g., SiO₂) and/or a | | | | | | | | |
|---|---|---|---|---|---|---|---|---|---|
| | semiconductor (e.g., Si, GaN, AlN, TiO₂, ZnO, SiC, copper oxide, cobalt oxide) | | | | | | | | |
| | 1A | | | | | | | | 3E |
| | Metal- | 1B | | | | | | | Metal- |
| | containing | Metal- | 2A | 2B | | | | | containing |
| | particle | containing | Surface | Surface | 3A | 3B | 3C | 3D | particles |
| | size | particle | area/mass ratio | area/mass ratio | Metal- | Metal- | Metal- | Metal- | comprising a |
| | about | size about | of at | of at | containing | containing | containing | containing | metal |
| Hydrogen- | 2 nm to | 2 nm to | least | least | particles | particles | particles | particles | alloy, e.g., |
| containing | about | about | about | about | comprising | comprising | comprising | comprising | Pt—Pd or |
| molecular species | 100 nm | 15 nm | 50 m²/g | 100 m²/g | Pt | Ru | Pd | Rh | Pt—Rh |
| Cycloalkanes | X | X | X | X | X | X | X | X | X |
| Cyclohexane | X | X | X | X | X | X | X | X | X |
| Methylcyclohexane | X | X | X | X | X | X | X | X | X |
| Decalin (cis- or | X | X | X | X | X | X | X | X | X |
| trans-decalin) | | | | | | | | | |
| Any compound | X | X | X | X | X | X | X | X | X |
| shown in FIG. 4 | | | | | | | | | |

In those variations in which the R—XH molecules interact with the nanostructured catalyst to form a species bound with the metal or metal alloy nanoparticles, the bound species may be dissociated from the nanostructured catalyst to produce an unbound dehydrogenated (“spent”) compound. As used herein, “dehydrogenated” is meant to encompass fully dehydrogenated as well as partially dehydrogenated. In some variations, a dehydrogenated species corresponding to the hydrogenated species R—XH may have the formula R—X—X—R, e.g., a disulfide. In other cases, a dehydrogenated species may comprise multiple R—X groups dissociated from the nanostructured catalyst. In still other variations, a dehydrogenated species may be a cyclic species with an X—X bond, e.g., a cyclic disulfide, such as 1,2-dithiolane.

Any suitable method may be used for dissociating a bound R—X species from the nanostructured catalysts. In general, enough energy may be applied to disrupt an X-Metal interaction formed between the R—X species and the metal or metal alloy nanoparticles in the nanostructured catalyst, e.g., at least about 10 kJ/mol, at least about 20 kJ/mol, at least about 30 kJ/mol, at least about 40 kJ/mol, or at least about 50 kJ/mol. For example, an Au—S interaction may be disrupted by applying about 35 kJ/mol. The energy may be delivered in any suitable manner, e.g., through the application of heat, electrical current, and/or light (e.g., ultraviolet light). For example, in some variations, application of heat so that the catalyst reaches at least about 50° C., at least about 60° C., at least about 70° C., at least about 80° C., at least about 90° C., or at least 100° C. may be sufficient to dissociate a bound species from a nanostructured catalyst described here.

The nanostructured catalysts described herein may be used to rehydrogenate spent compounds with hydrogen to regenerate the hydrogen-containing molecular species to store that hydrogen, so that it is available for use upon catalytic dehydrogenation as described above.

As is shown in Tables III and IV following, various combinations of nanostructured catalysts may be used with each spent compound. In each of the variations shown in Tables III and IV, if present the nanostructures in the nanostructured mesh or mat may have a cross-sectional dimension of about 5 nm to about 500 nm, e.g., about 5 nm to about 300 nm, or about 5 nm to about 200 nm, or about 5 nm to about 100 nm, or about 15 nm to about 100 nm. In Tables III and IV, a nanostructured catalyst that may be used in combination with any of the hydrogen-containing molecular species compound may include any feature in column 1A in combination with any feature in column 2A, column 2B, column 3A, column 3B, column 3C, column 3D, column 3E, or column 3F. Further, a nanostructured catalyst may include any feature in column 1B in combination with any feature in column 2A, column 2B, column 3A, column 3B, column 3C, column 3D, column 3E, or column 3F, a nanostructured catalyst may include any feature in column 2A in combination with any feature in column 1A, column 1B, column 3A, column 3B, column 3C, column 3D, column 3E, or column 3F, a nanostructured catalyst may include any feature in column 2B in combination with any feature in column 1A, column 1B, column 3A. column 3B, column 3C, column 3D, column 3E, or column 3F, a nanostructured catalyst may include any feature in column 3A in combination with any feature in column 1A, column 1B, column 2A, or column 2B, a nanostructured catalyst may include any feature in column 3B in combination with any feature in column 1A, column 1B, column 2A, or column 2B, a nanostructured catalyst may include any feature in column 3C in combination with any feature in column 1A, column 1B, column 2A, or column 2B, a nanostructured catalyst may include any feature in column 3D in combination with any feature in column 1A, column 1B, column 2A, or column 2B, a nanostructured catalyst may include any feature in column 3E in combination with any feature in column 1A, column 1B, column 2A, or column 2B, and a nanostructured catalyst may include any feature in column 3F in combination with any feature in column 1A, column 1B, column 2A, or column 2B. The nanostructures used in the catalysts may comprise an insulator, e.g., SiO₂, and/or a semiconductor, e.g., GaN nanowires or a GaN coating on SiO₂ nanostructures, and the nanoparticles may be e.g., Au in each of the combinations disclosed in Table III. The nanostructures used in the catalysts may comprise an insulator, e.g., SiO₂, and/or a semiconductor, e.g., GaN nanowires or a GaN coating on SiO₂ nanostructures, and the nanoparticles may be e.g., Pt in each of the combinations disclosed in Table IV. Thus, for example, any dehydrogenated product from an R—XH molecule, e.g., R—S—S—H or 1,2-dithiolane, may be used with a nanostructured catalyst comprising GaN decorated with gold nanoparticles having a dimension of about 2 nm to about 15 nm and a surface/mass ratio of at least about 50 m²/g. In some variations, 1,4-cyclohexanedithiol may be used in combination with a nanostructured catalyst comprising a disordered array of GaN nanowires having a dimension of about 10 nm to about 500 nm decorated with gold nanoparticle having a dimension of about 2 nm to about 15 nm, and a surface/mass ratio of about 60 m²/g, about 70 m²/g, about 80 m²/g, about 90 m²/g, about 100 m²/g, about 110 m²/g, about 120 m²/g, about 130 m²/g, about 140 m²/g, or about 150 m²/g.

TABLE III

| | | Nanostructured catalyst comprising a substrate and/or nanostructures comprising | | | | | | | | | |
|---|---|---|---|---|---|---|---|---|---|---|---|
| | | an insulator (e.g., SiO₂) and/or a semiconductor (e.g., Si, GaN, AlN, | | | | | | | | | |
| | | TiO₂, ZnO, SiC, copper oxide, cobalt oxide) | | | | | | | | | |
| | | 1A | 1B | | | | | | | | |
| | | Metal- | Metal- | 2A | 2B | 3A | 3B | 3C | 3D | 3E | 3F |
| | | containing | containing | Surface | Surface | Metal-containing particles comprising: | | | | | |
| | | particle | particle | area/mass | area/mass | | | | | | Metal |
| | | size about | size about | ratio of at | ratio of at | | | | | | alloys, |
| Dehydrogenated | | 2 nm to | 2 nm to | least | least | | | | | | e.g., |
| compound (spent | | about | about | about | about | | | | | Pd, Ru, | Pt—Pd, |
| compound) | | 100 nm | 15 nm | 50 m²/g | 100 m²/g | Au | Ag | Pt | Cu | or Rh | or Pd—Rh |
| Dehydrogenated | | X | X | X | X | X | X | X | X | X | X |
| compounds | | | | | | | | | | | |
| corresponding to R₁—X, | | | | | | | | | | | |
| where X = S, Se, or | | | | | | | | | | | |
| O, e.g., R₁—X—X—R₁, | | | | | | | | | | | |
| or R₂—XH, where R₂ | | | | | | | | | | | |
| is dehydrogenated | | | | | | | | | | | |
| relative to R₁, or | | | | | | | | | | | |
| R₃═X, where R₃ is | | | | | | | | | | | |
| dehydrogenated | | | | | | | | | | | |
| relative to R₁ | | | | | | | | | | | |
| R₁, | Alkyl, e.g., | X | X | X | X | X | X | X | X | X | X |
| R₂, | straight or | | | | | | | | | | |
| or | branched | | | | | | | | | | |
| R₃ | C1-C20, e.g., | | | | | | | | | | |
| | methyl, | | | | | | | | | | |
| | propyl, | | | | | | | | | | |
| | butyl, pentyl, | | | | | | | | | | |
| | hexyl, | | | | | | | | | | |
| | heptyl, or | | | | | | | | | | |
| | octyl | | | | | | | | | | |
| | Heteroalkyl, | X | X | X | X | X | X | X | X | X | X |
| | e.g., alkyl | | | | | | | | | | |
| | group with | | | | | | | | | | |
| | one or more | | | | | | | | | | |
| | N, S, O, or | | | | | | | | | | |
| | Se | | | | | | | | | | |
| | heteroatoms | | | | | | | | | | |
| | Alkenyl, | X | X | X | X | X | X | X | X | X | X |
| | e.g., straight | | | | | | | | | | |
| | or branched | | | | | | | | | | |
| | C2-C20, e.g., | | | | | | | | | | |
| | C2-C12 | | | | | | | | | | |
| | Substituted | X | X | X | X | X | X | X | X | X | X |
| | alkenyl | | | | | | | | | | |
| | Alkynyl, | X | X | X | X | X | X | X | X | X | X |
| | e.g., straight | | | | | | | | | | |
| | or branched | | | | | | | | | | |
| | C2-C20, e.g., | | | | | | | | | | |
| | C2-12 | | | | | | | | | | |
| | Aryl, e.g., | X | X | X | X | X | X | X | X | X | X |
| | aromatic | | | | | | | | | | |
| | rings | | | | | | | | | | |
| | including | | | | | | | | | | |
| | about 6 to | | | | | | | | | | |
| | about 14 C | | | | | | | | | | |
| | atoms | | | | | | | | | | |
| | Heteroaryl, | X | X | X | X | X | X | X | X | X | X |
| | e.g., | | | | | | | | | | |
| | aromatic | | | | | | | | | | |
| | rings | | | | | | | | | | |
| | including | | | | | | | | | | |
| | one or more | | | | | | | | | | |
| | N, O, S, or | | | | | | | | | | |
| | Se | | | | | | | | | | |
| | heteroatoms | | | | | | | | | | |
| | and about 2 | | | | | | | | | | |
| | to about 14 | | | | | | | | | | |
| | C atoms | | | | | | | | | | |
| | Alkoxy, e.g., | X | X | X | X | X | X | X | X | X | X |
| | CH₃O—, | | | | | | | | | | |
| | C₂H₃O—, | | | | | | | | | | |
| | C₃H₇O—, | | | | | | | | | | |
| | C₄H₉O—, | | | | | | | | | | |
| | C₅H₁₁O—, or | | | | | | | | | | |
| | C₆H₁₃O— | | | | | | | | | | |
| | Cycloalkyl, | X | X | X | X | X | X | X | X | X | X |
| | e.g., | | | | | | | | | | |
| | cyclopropyl, | | | | | | | | | | |
| | cylcobutyl, | | | | | | | | | | |
| | cyclopentyl, | | | | | | | | | | |
| | cyclohexyl, | | | | | | | | | | |
| | cycloheptyl, | | | | | | | | | | |
| | or cyclooctyl | | | | | | | | | | |
| | Heterocyclic | X | X | X | X | X | X | X | X | X | X |
| | e.g., cyclic | | | | | | | | | | |
| | groups with | | | | | | | | | | |
| | 1 or more N, | | | | | | | | | | |
| | O, S, or Se | | | | | | | | | | |
| | atoms; e.g., | | | | | | | | | | |
| | 3, 4, 5, 6, 7, | | | | | | | | | | |
| | 8, 9, 10, 11, | | | | | | | | | | |
| | 12, 13 or 14 | | | | | | | | | | |
| | membered | | | | | | | | | | |
| | rings | | | | | | | | | | |
| | including a | | | | | | | | | | |
| | heteroatom. | | | | | | | | | | |
| | Alkyl- | X | X | X | X | X | X | X | X | X | X |
| | substituted | | | | | | | | | | |
| | aryl groups | | | | | | | | | | |
| | Aryl- | X | X | X | X | X | X | X | X | X | X |
| | substituted | | | | | | | | | | |
| | alkyl groups | | | | | | | | | | |
| | Aryl- | X | X | X | X | X | X | X | X | X | X |
| | substituted | | | | | | | | | | |
| | alkenyl | | | | | | | | | | |
| | groups | | | | | | | | | | |
| | Aryl- | X | X | X | X | X | X | X | X | X | X |
| | substituted | | | | | | | | | | |
| | alkynyl | | | | | | | | | | |
| | groups | | | | | | | | | | |
| | Arylenes, | X | X | X | X | X | X | X | X | X | X |
| | including | | | | | | | | | | |
| | divalent | | | | | | | | | | |
| | aromatic | | | | | | | | | | |
| | groups | | | | | | | | | | |
| | including | | | | | | | | | | |
| | about 6 up to | | | | | | | | | | |
| | about 14 C | | | | | | | | | | |
| | atoms | | | | | | | | | | |
| | Oxyarylene | X | X | X | X | X | X | X | X | X | X |
| | Substituted | X | X | X | X | X | X | X | X | X | X |
| | arylenes | | | | | | | | | | |
| | 1,2- | X | X | X | X | X | X | X | X | X | X |
| | Dithiolane | | | | | | | | | | |
| | Dithioparabenzoquinone | X | X | X | X | X | X | X | X | X | X |
| | 1,4- | X | X | X | X | X | X | X | X | X | X |
| | Benzenedithiol | | | | | | | | | | |
| | Dehydrogenated | X | X | X | X | X | X | X | X | X | X |
| | form of | | | | | | | | | | |
| | any | | | | | | | | | | |
| | compound in | | | | | | | | | | |
| | FIG. 3 | | | | | | | | | | |

| | | | | | | | | | | |
|---|---|---|---|---|---|---|---|---|---|---|
| Dehydrogenated | X | X | X | X | X | X | X | X | X | X |
| form of any | | | | | | | | | | |
| cycloalkane | | | | | | | | | | |
| Dehydrogenated | X | X | X | X | X | X | X | X | X | X |
| form of any | | | | | | | | | | |
| compound in FIG. 4 | | | | | | | | | | |
| Benzene | X | X | X | X | X | X | X | X | X | X |
| Toluene | X | X | X | X | X | X | X | X | X | X |
| Naphthalene | X | X | X | X | X | X | X | X | X | X |

TABLE IV

| | Nanostructured catalyst comprising a substrate and/or nanostructures | | | | | | | | |
|---|---|---|---|---|---|---|---|---|---|
| | comprising an insulator (e.g., SiO₂) and/or a semiconductor | | | | | | | | |
| | (e.g., Si, GaN, AlN, TiO₂, ZnO, SiC, copper oxide, cobalt oxide) | | | | | | | | |
| | 1A | 1B | | | | | | | 3E |
| | Metal- | Metal- | 2A | 2B | 3A | 3B | 3C | 3D | Metal-containing |
| | containing | containing | Surface | Surface | Metal- | Metal- | Metal- | Metal- | particles |
| Dehydrogenated | particle size | particle size | area/mass | area/mass | containing | containing | containing | containing | comprising |
| compound | about | about 2 nm | ratio of at | ratio of at | particles | particles | particles | particles | a bimetal, |
| (spent | 2 nm to about | to about | least about | least about | comprising | comprising | comprising | comprising | e.g., Pt—Pd |
| compound) | 100 nm | 15 nm | 50 m²/g | 100 m²/g | Pt | Ru | Pd | Rh | or Pt—Rh |
| Dehydrogenated | X | X | X | X | X | X | X | X | X |
| form of any | | | | | | | | | |
| cycloalkane | | | | | | | | | |
| Dehydrogenated | X | X | X | X | X | X | X | X | X |
| form of any | | | | | | | | | |
| compound in | | | | | | | | | |
| FIG. 4 | | | | | | | | | |
| Benzene | X | X | X | X | X | X | X | X | X |
| Toluene | X | X | X | X | X | X | X | X | X |
| Naphthalene | X | X | X | X | X | X | X | X | X |

Examples of particular methods to which a nanostructured catalyst may be applied include:
1. A method of generating hydrogen, the method comprising:

providing a nanostructured catalyst comprising metal-containing nanoparticles disposed on a substrate and/or on a disordered array of nanostructures;

reacting a compound capable of generating hydrogen and having a formula R₁—XH with the nanostructured catalyst to produce hydrogen gas and R₁—X bound to the nanostructured catalyst and/or a dehydrogenated spent compound; and

collecting the hydrogen gas,

wherein:

R₁ is a moiety selected from the group consisting of an alkyl, a heteroalkyl, an alkenyl, a substituted alkenyl, an alkynyl, an aryl, a heteroaryl, an alkoxy, a cycloalkyl, a heterocyclic, an alkylaryl, an arylalkyl, an arylalkenyl, an arylalkynyl, an arylene, an oxyarylene group, and combinations thereof; and

X is selected from the group consisting of sulfur, oxygen and selenium.
2. The method of paragraph 1 above, wherein the substrate and/or at least some of the nanostructures in the disordered array comprise a semiconductor.
3. The method as in paragraph 1 or 2, wherein the substrate and/or at least some of the nanostructures in the disordered array comprise at least one of the group consisting of GaN, Si, SiC, TiO₂, ZnO, AlN, copper oxide and cobalt oxide.
4. The method as in any of paragraphs 1-3, wherein at least some of the nanostructures in the disordered array comprise SiO₂.
5. The method of paragraph 4, wherein the disordered array of nanostructures comprises silica nanostructures at least partially coated with a semiconductor.
6. The method of paragraph 2, wherein the substrate and/or disordered array of nanostructures comprises a region comprising polycrystalline GaN.
7. The method of paragraph 2, wherein the substrate and/or disordered array of nanostructures comprises a region comprising single crystal GaN.
8. The method as in any of paragraphs 1-7, wherein the nanoparticles comprise a metal selected from the group consisting of Au, Ag, Cu, Pd, Pt, Ru, Rh, Fe, Ni, Co and alloys and combinations thereof.
9. The method as in any of paragraphs 1-8, wherein a surface area to mass ratio of the nanoparticles on the substrate and/or nanostructures is at least about 50 m²/g.
10. The method as in any of paragraphs 1-9, wherein disordered array comprises nanostructures having an average cross-sectional dimension in a range from about 5 nm to about 500 nm.
11. The method as in any of paragraphs 1-9, wherein the disordered array comprises nanostructures having an average cross-sectional dimension in a range from about 5 nm to about 100 nm.
12. The method as in any of paragraphs 1-11, wherein the nanostructured catalyst comprises nanoparticles having an average cross-sectional dimension in a range from about 2 nm to about 100 nm.
13. The method of paragraph 12, wherein the nanostructured catalyst comprises nanoparticles having an average cross-sectional dimension in a range from about 2 nm to about 15 nm.
14. The method as in any of paragraphs 1-13, wherein the nanoparticles are distributed on the substrate and/or nanostructures such that the majority of the nanoparticles are generally isolated from each other.
15. The method of paragraph 14, wherein the nanoparticles are distributed on the substrate and/or nanostructures such that there is physical contact between at most about 30% of the nanoparticles.
16. The method as in any of paragraphs 1-15, wherein the disordered array of nanostructures comprises nanowires, nanosprings, nanorods, nanotubes, or a combination thereof.
17. The method of paragraph 16, wherein the substrate and/or disordered array of nanostructures comprises GaN, and the nanoparticles disposed on the substrate and/or disordered array of nanostructures comprise Au.
18. The method as in any of paragraphs 1-17, comprising reacting the nanostructured catalyst with a compound having the formula R₁—SH.
19. The method of paragraph 18, wherein R₁ is a C2-C8 alkyl, heteroalkly, alkenyl, or heteroalkenyl group.
20. The method as in any of paragraphs 1-17, comprising reacting the nanostructured catalyst with a C2-C8 organothiol.
21. The method as in any of paragraphs 1-17, comprising reacting the nanostructured catalyst with 1,4-cyclohexanedithiol.
22. The method as in any of paragraphs 1-21, wherein collecting the hydrogen gas comprises consuming the hydrogen gas in an engine or fuel cell.

### IV. Examples

Examples 1 through 6 provide examples of various nanostructured catalysts that may be used for various catalytic reactions, and Example 7 provides an example of a reaction that was catalyzed by a nanostructured catalyst described here. For example, any of the Examples 1 through 4 may be used for a water gas shift reaction, or combined with any one of the hydrogen-containing molecular species shown in Tables I and II above to generate hydrogen gas for fuel. Examples 5 and 6 provide examples of various nanostructured catalysts that may be used to catalyze other types of reactions, e.g., a polymerization reaction.

For those examples in which hydrogen gas is generated, it should be pointed out that the hydrogen gas may be produced and immediately collected by an energy-consuming device or system, e.g., an engine or a fuel cell. In those variations, the nanostructured catalyst may be integral with or connected to the energy-consuming device or system. In other variations, the nanostructured catalyst may be separate from an energy-consuming device or system so that the hydrogen generated is collected and later delivered to the energy-consuming device or system. Further, these and similar variations of nanostructured catalysts may be used with any one of the dehydrogenated molecular species shown in Tables III and IV above and hydrogen gas to store hydrogen for future use. Here again, the nanostructured catalyst device for rehydrogenation of spent compounds may be integral with or connected to an energy-consuming system so that the spent compounds can be locally rehydrogenated, leading to local storage of hydrogen. In other variations, the nanostructured catalyst device for rehydrogenation of spent compounds may be separate from an energy-consuming device or system.

The hydrogen-containing molecule may be a liquid, e.g., a liquid organothiol or a o liquid cycloalkane, and injected into a chamber comprising the nanostructured catalyst. The hydrogen-containing molecule may then be heated sufficiently to vaporize the molecule (e.g., heated to about 200° C.). The vaporized hydrogen-containing molecule can then be adsorbed onto the nanostructured catalyst, whereby hydrogen gas is released. The hydrogen gas may be collected and used, e.g., consumed by an engine or the like, and/or collected and stored.

### Example 1

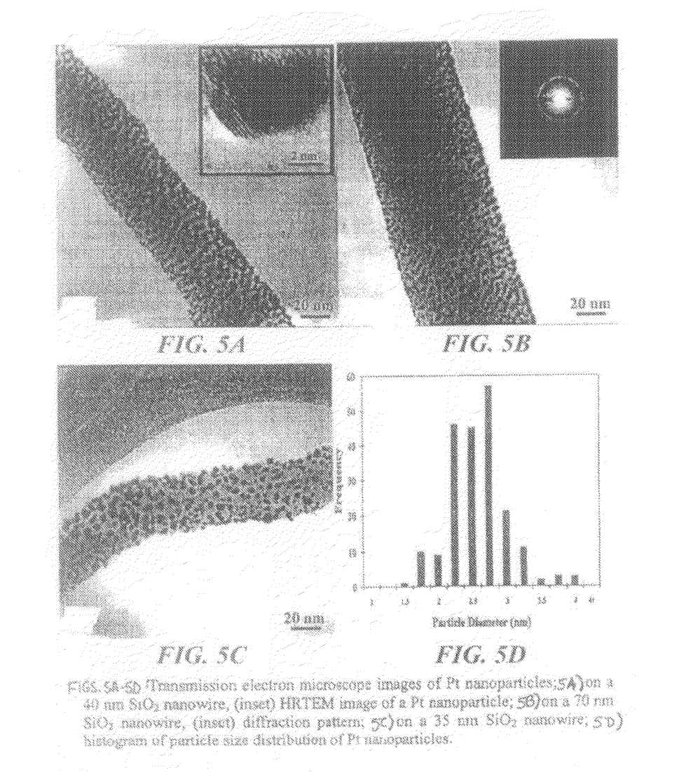

Referring now to FIGS. 5A-5C, transmission electron microscopy images are provided for one variation of a nanostructured catalyst that may be used with a reaction that can be catalyzed by platinum, e.g., any one of the hydrogen-containing molecular species disclosed herein or known in the art to generate hydrogen gas and/or rehydrogenate any dehydrogenated molecular species disclosed herein or known in the art to store hydrogen gas.

The variation of nanostructured catalyst illustrated in FIGS. 5A-5C includes SiO₂ nanowires that have been grown on a silicon substrate. In this particular variation, Pt nanoparticles have been deposited on the surface of the SiO₂ nanowires using chemical vapor deposition. FIG. 5D shows a histogram graph showing a particle size distribution of the Pt nanoparticles. As is seen, an average particle size is between about 2 nm and about 3 nm. FIG. 5A shows Pt nanoparticles on an approximately 40 nm diameter SiO₂ nanowire (the inset shows a high resolution TEM image), FIG. 5B shows Pt nanoparticles on an approximately 70 nm diameter SiO₂ nanowire (the inset shows a diffraction pattern showing that the Pt is crystalline), and FIG. 5C shows Pt nanoparticles on an approximately 35 nm diameter SiO₂ nanowire. In this example, the SiO₂ nanowires have been grown from a silicon substrate using a 30 nm Au catalytic layer and dimethylsilane as the starting material, using the methods described in International Patent Application No. PCT/US2006/024435, “Method for Manufacture and Coating of Nanostructured Components,” filed Jun. 23, 2006, which has already been incorporated by reference herein in its entirety. The Pt nanoparticles were formed on the substrate and the nanowires by delivering solid dimethyl(1,5-cyclooctadiene)platinum(II) [(CH₃)₂Pt(C₈H₁₂)] by sublimation to a growth chamber by heating to 343K in an argon stream flowing at 10 standard cubic cm per minute (sccm). The process time was about 12 minutes. In a range of experiments, the total pressure in the growth chamber was varied at pre-determined settings of 17, 42, and 67 Pa. The temperature of the substrate including the SiO₂ nanowires was also varied at fixed values of 573K, 723K, and 873K. In this example of a nanostructured catalyst, the metal Pt nanoparticles are present at such a density on the surface of the nanowire that the nanoparticles are generally isolated from each other. Thus, in general about 30% or less, or about 20% or less of the Pt nanoparticles are in physical contact with each other. The coverage of the underlying nanowire may in general not be complete. In some variations, the coverage of the Pt nanoparticles on the SiO₂ nanowire surface may be about 50% to about 90%. In other variations, the coverage of the nanoparticles on the nanowire surface may be at or close to 100%. In some instances, adjacent or abutting nanoparticles may be separated by a boundary, e.g., lattice planes across the boundary may not be continuous, there may be a rotation or twist of the lattice planes across the boundary, and/or the lattice planes may be separated by a facet plane at the boundary.

As described herein, any one of or any combination of the following variables for the catalyst illustrated in FIGS. 5A-5D may be varied to change the performance of the catalyst for catalyzing a particular reaction: i) the Pt particle size; ii) the Pt particle distribution on the SiO₂ nanosprings; iii) the density of SiO₂ nanosprings; iv) the diameter or cross-sectional shape of the nanosprings; and v) whether a semiconductor layer (such as ZnO or TiO₂) is provided over the SiO₂ nanostructures and the Pt nanoparticles are deposited on the semiconductor layer.

### Example 2

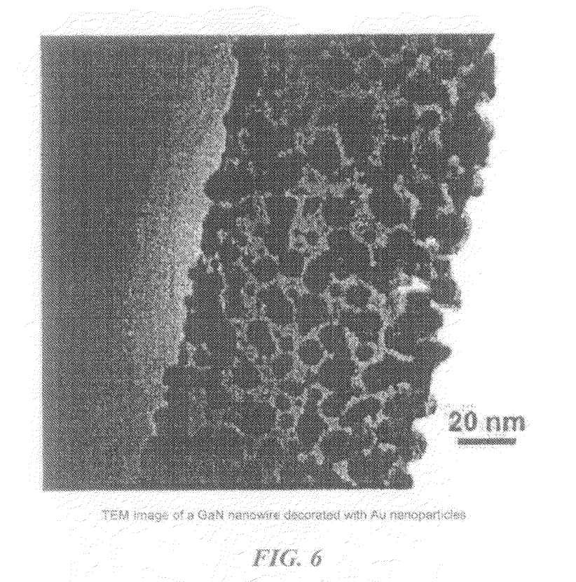

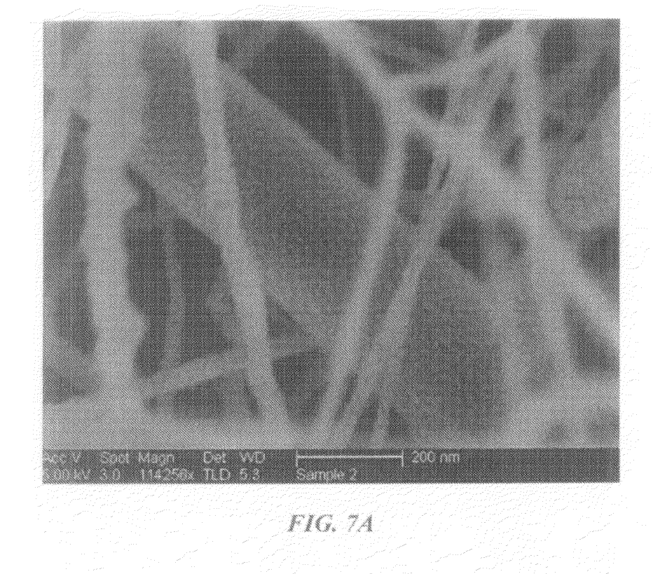

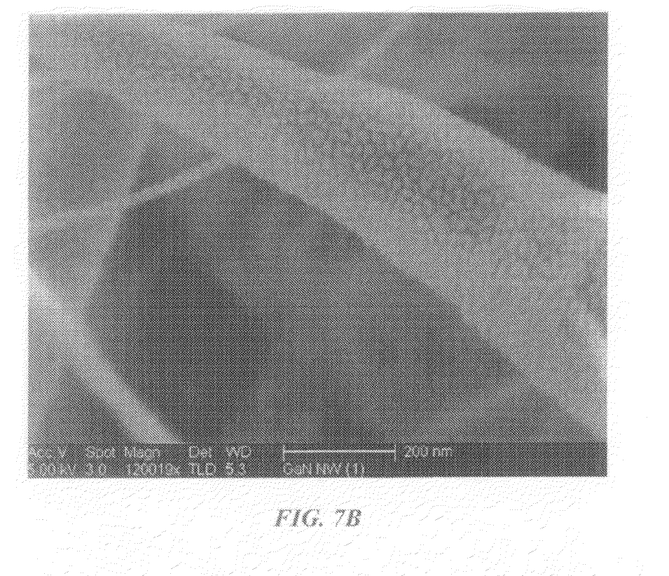

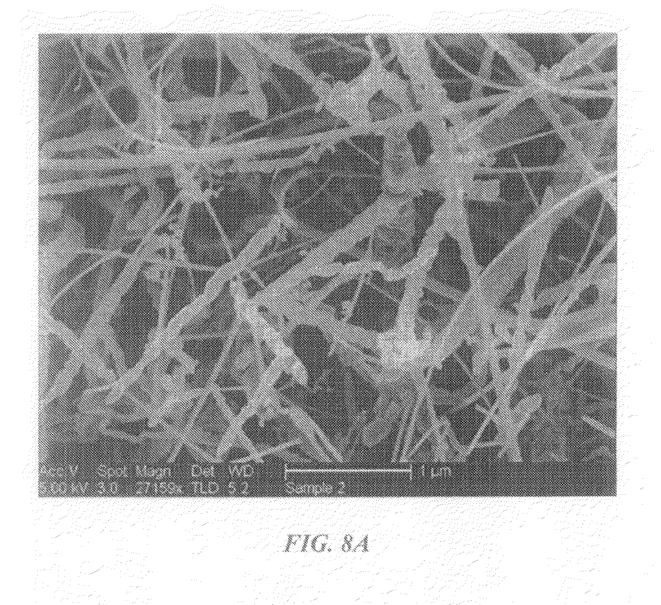

Another example of a nanostructured catalyst is shown by transmission electron microscopy image in FIG. 6. In this example, a GaN nanowire has been coated with Au nanoparticles. This type of catalyst may be used in a water gas shift reaction. Also, this variation of nanostructured catalyst may be used with any one of the hydrogen-containing molecular species shown in Tables I and II above to generate hydrogen gas for use as an energy source. Further, this and similar variations of nanostructured catalysts may be used with any one of the dehydrogenated molecular species shown in Tables III and IV above and hydrogen gas to store hydrogen for future use. For example, this nanostructured catalyst may be used to catalytically release hydrogen gas stored in molecules having the formula R—XH, e.g., R—SH molecules such as 1,4-cyclohexanedithiol. Alternatively or in addition, this nanostructured catalyst may be used to react a dehydrogenated molecule with hydrogen gas to store hydrogen for later use, e.g., it may be used to react dithioparabenzoquinone or 1,4-benzenedithiol with hydrogen gas to form 1,4, cyclohexanedithiol. The regenerated 1,4-cyclohexanedithiol may then be used to store the hydrogen until the hydrogen is needed as an energy source, when it can be catalytically released using a nanostructured catalyst described herein.

In this example, the GaN nanowire has been grown from a sapphire substrate using a 60 nm Ni catalytic layer and NH₃ and Ga as starting materials, using the methods described in International Patent Application No. PCT/US2006/024435, “Method for Manufacture and Coating of Nanostructured Components,” filed Jun. 23, 2006, which has already been incorporated by reference herein in its entirety. The Au nanoparticles were deposited on the GaN nanowires at 573K, at a pressure of 300 mTorr, and using dimethyl(acetylacetonate)gold(III) as the starting material. The GaN nanowires may comprise one or more regions of polycrystalline GaN and/or one or more regions of single crystal GaN. Here again, the coverage of the Au nanoparticles on the GaN underlying nanostructure is not complete, so that the Au nanoparticles may be generally isolated from each other, and a portion of the underlying GaN structure is exposed.

The hydrogen-containing molecule may be a liquid, e.g., a liquid organothiol or a liquid cycloalkane, and injected into a chamber comprising the nanostructured catalyst. The hydrogen-containing molecule may then be heated sufficiently to vaporize the molecule (e.g., heated to about 200° C.). The vaporized hydrogen-containing molecule can then be adsorbed onto the nanostructured catalyst, whereby hydrogen gas is released. The hydrogen gas may be collected and used, e.g., consumed by an engine or the like, and/or collected and stored.

For the catalyst example illustrated in FIG. 6, any one or any combination of the following variables may be changed to adjust the performance of the catalyst for a particular reaction: i) the Au particle size; ii) the Au particle distribution on the GaN nanostructures; iii) the density of GaN nanostructures; iv) the diameter or cross-sectional shape of the nanostructures; and v) the nature of the crystallinity (the relative distributions of polycrystalline regions and single crystal regions in the GaN).

### Example 3

Referring now to FIGS. 7A-7B, another variation of a nanostructured catalyst is illustrated. This type of catalyst may be used in a water gas shift reaction, and also may be used to catalytically release hydrogen gas from any hydrogen-containing molecule described herein or known in the art, or to react a spent compound with hydrogen gas to rehydrogenate the spent compound to regenerate a hydrogen-containing molecule that may be used to store hydrogen for later use. The variation of the nanostructured catalyst shown in FIGS. 7A-7B may for example be used to release hydrogen gas stored in any of the hydrogen-containing molecules shown in Tables I and II above, e.g., an organothiol compound such as 1,4-cyclohexanedithiol. Alternatively or in addition, the variation of the nanostructured catalyst shown in FIGS. 7A-7B may be used to react hydrogen gas with a spent compound to form a hydrogen-containing molecule that may be used to store hydrogen for later catalytic release. For example, this variation of nanostructured catalyst may be used to regenerate 1,4-cyclohexanethiol from 1,4-benzenedithiol or dithioparabenzoquinone.

The nanostructured catalyst shown in FIGS. 7A-7B comprises a disordered array of GaN nanostructures. In this example, the GaN nanowires have been grown from a sapphire substrate using a 60 nm Ni catalytic layer and NH₃ and Ga as starting materials, using the methods described in International Patent Application No. PCT/US2006/024435, “Method for Manufacture and Coating of Nanostructured Components,” filed Jun. 23, 2006, which has already been incorporated by reference herein in its entirety. The Au nanoparticles were deposited on the GaN nanowires at 573 K, at a pressure of 300 mTorr, and using dimethyl(acetylacetonate)gold(III) as a starting material. The GaN nanowires may comprise one or more regions of polycrystalline GaN and/or one or more regions of single crystal GaN. As is seen in this variation, the disordered array of nanostructures comprises nanostructures with widely varying cross-sectional dimensions. For example, FIGS. 7A-7B show a nanowire having a cross-sectional diameter of greater than about 200 nm in the same disordered array as a nanowire having a cross-sectional diameter of about 10 nm or even less. The Au nanoparticles are present at a sufficient density so that the majority of the nanowire surface is coated, but so that the nanoparticles are generally isolated from each other. Here, it appears that the Au nanoparticle coverage of the GaN nanowire surface is at least about 90%; however, distinct boundaries between Au nanoparticles are clearly present. As stated above, the boundaries between Au nanoparticles may be disruptions in lattice planes between adjacent particles, e.g., the lattice planes across the boundary may not be continuous, there may be a rotation or twist of the lattice planes across a boundary and/or the lattice planes may be separated by a facet plane at the boundary. The Au nanoparticles individually also have some surface structures, e.g., they may include sub nanometer features which may contribute to their catalytic activity.

For the catalyst example illustrated in FIGS. 7A-7B, any one or any combination of the following variables may be changed to adjust the performance of the catalyst for a particular reaction: i) the Au particle size; ii) the Au particle distribution on the GaN nanostructures; iii) the density of GaN nanostructures; iv) the diameter or cross-sectional shape of the nanostructures; and v) the nature of the crystallinity (the relative distributions of polycrystalline regions and single crystal regions in the GaN).

### Example 4

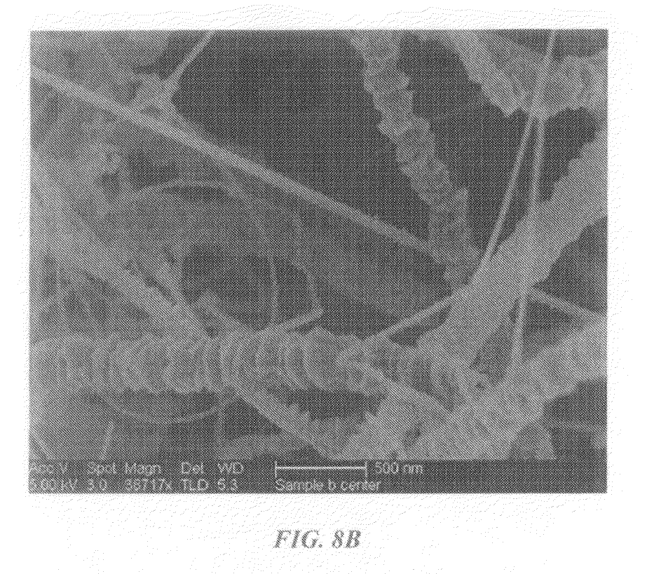

FIGS. 8A-8B illustrate another variation of a nanostructured catalyst. This variation of catalyst may for example be used to catalyze a water gas shift reaction, or may be used to catalytically release hydrogen gas from any hydrogen-containing molecule described herein or known in the art, or to react a spent compound with hydrogen gas to rehydrogenate the spent compound to regenerate a hydrogen-containing molecule that may be used to store hydrogen for later use. The variation of the nanostructured catalyst shown in FIGS. 8A-8B may for example be used to release hydrogen gas stored in any of the hydrogen-containing molecules shown in Tables I and II above, e.g., an organothiol compound such as 1,4-cyclohexanedithiol. Alternatively or in addition, the variation of the nanostructured catalyst shown in FIGS. 8A-8B may be used to react hydrogen gas with a spent compound to form a hydrogen-containing molecule that may be used to store hydrogen for later catalytic release. For example, this variation of nanostructured catalyst may be used to regenerate 1,4-cyclohexanethiol from 1,4-benzenedithiol or dithioparabenzoquinone.

The particular variation of nanostructured catalyst illustrated in FIGS. 8A-8B comprises a disordered array of GaN nanowires grown on a sapphire substrate. In this example, the GaN nanowires have been grown from a sapphire substrate using a 60 nm Ni catalytic layer and NH₃ and Ga as starting materials, using the methods described in International Patent Application No. PCT/US2006/024435, “Method for Manufacture and Coating of Nanostructured Components,” filed Jun. 23, 2006, which has already been incorporated by reference herein in its entirety. The Au nanoparticles were deposited on the GaN nanowires at 573 K, at a pressure of 300 mTorr, and using dimethyl(acetylacetonate)gold(III) as a starting material. In this variation, the GaN nanowires exhibit a variety of morphologies. For example, the relatively larger, irregular, structures likely comprise polycrystalline GaN nanowires and the relatively smaller, smoother, structures likely comprise single crystalline GaN regions. FIG. 8B shows an expanded view of Au nanoparticles on a polycrystalline GaN nanowire. As shown in FIGS. 8A and 8B, GaN nanowires having a cross-sectional dimension of about 300 nm are formed in the same disordered array with nanowires having a cross-sectional dimension of less than about 10 nm. Here, the Au nanoparticles coverage the majority of the GaN underlying nanostructure (e.g., greater than about 90% of the GaN surface area). However, the individual Au nanoparticles may be generally isolated from each other, or individual Au nanoparticles may abut each other but be separated by a boundary, e.g., a disruption between lattice planes as described above. In some variations, a portion of the underlying GaN surface nanostructure may be exposed.

The nanostructured catalyst shown in FIGS. 8A-8B may be used in combination with hydrogen-containing molecules shown in Table I to generate hydrogen gas with high efficiency and at high rates. For example, a liquid organothiol, e.g., 1,4-cyclohexanedithiol, may be injected into a chamber containing the nanostructured catalyst shown in FIGS. 8A-8B and heated to about 200° C. so as to vaporize the organothiol. The organothiol molecule may then be adsorbed onto any portion of the surface of the nanostructured catalyst to release hydrogen. For example, the organothiol molecule may be adsorbed onto the Au nanoparticles on the disordered array and/or onto a residual catalytic layer of gold left on the sapphire substrate.

For the catalyst example illustrated in FIGS. 8A-8B, any one or any combination of the following variables may be changed to adjust the performance of the catalyst for a particular reaction: i) the Au particle size; ii) the Au particle distribution on the GaN nanostructures; iii) the density of GaN nanostructures; iv) the diameter or cross-sectional shape of the nanostructures; and v) the nature of the crystallinity (the relative distributions of polycrystalline regions and single crystal regions in the GaN).

### Example 5

Referring now to FIGS. 9A-9C, an example of a nanostructured catalyst comprising SiO₂ nanostructures and ZnO nanoparticles is illustrated. In this particular variation, the disordered array 900 is formed from a plurality of coiled nanostructures 920. Platelet-like ZnO nanoparticles 930 are disposed on the surface of the nanostructures 920. The coiled nanostructures have a range of cross-sectional diameters, a range of lengths, a range of pitches in the helical coils, and there is no apparent order to the arrangement of the nanostructures. The resulting macroporous network comprises a wide size distribution and a wide spatial distribution of inter-structure volumes that can be accessible catalytic sites. In this particular variation, no metal nanoparticles are disposed over the ZnO particles; however, in other embodiments, metal nanoparticles can be distributed over the ZnO particles.

To form the nanostructured catalysts depicted in FIGS. 9A-9C, a disordered array of helical silica nanostructures was grown on a silicon substrate as in Example 1. Zinc oxide nanoparticles were deposited on the silica nanostructures using Atomic Layer Deposition (ALD). The precursor material used was diethyl zinc, which was introduced into a deposition chamber at a chamber pressure of about 1 Torr with the substrate at a temperature of about 200° C. Thereafter, the chamber was pumped out and purged with dry nitrogen. Water vapor was introduced into the chamber as an oxygen source at a chamber pressure of about 1 Torr with the substrate temperature still at about 200° C. Each cycle of zinc precursor and the oxygen source produce a single layer of zinc oxide. The deposition process was repeated for 200 cycles to produce a continuous layer of zinc oxide nanoparticles, where the average particle size was about 100 nm by about 10 nm. The zinc oxide nanoparticles deposited on the nanostructures are crystalline in Example 5, as determined by the observation of faceting in the SEM image (see FIGS. 9A-9C).

Nanostructured catalysts comprising zinc oxide nanoparticles deposited on silica nanostructures may be used to catalyze a number of reactions including the synthesis of nitriles from aldoximes, the acylation of alcohols, phenols, and amines, and β-acetamido ketones via condensation reactions.

### Example 6

FIGS. 10A-10B illustrate an example of a nanostructured catalyst comprising SiO₂ nanostructures with TiO₂ nanoparticles applied to the nanostructures. The disordered array 1000 comprises nanostructures 1020 coated with TiO₂ nanoparticles 1030.

As stated above, a catalyst similar to that illustrated in FIGS. 10A-10B may be useful in catalyzing the polymerization of ethylene or propylene, e.g., using conditions similar to those described in de Souza et al., Applied Catalysis A: General, 323 (2007), 234.

To form the nanostructured catalysts depicted in FIGS. 10A and 10B, a disordered array of helical silica nanostructures was grown on a silicon substrate as in Example 1. Titanium dioxide nanoparticles were deposited on the silica nanostructures using Atomic Layer Deposition (ALD). The precursor material used was titanium chloride, TiCl₄, which was introduced into a deposition chamber at a chamber pressure of about 1 Torr with the substrate at a temperature of about 300° C. Thereafter, the chamber was pumped out and purged with dry nitrogen. Water vapor was introduced into the chamber as an oxygen source at a chamber pressure of about 1 Torr with the substrate temperature still at about 300° C. Each cycle of titanium precursor and the oxygen source produce a single layer of titanium dioxide. The deposition process was repeated for 50 cycles to produce a continuous layer of titanium dioxide nanoparticles, where the. average particle size was about 200 nm in the longest dimension. The titanium dioxide nanoparticles deposited on the nanostructures are crystalline in Example 6, as determined by the observation of faceting in the SEM image (See FIGS. 10A and 10B).

### Example 7

GaN nanowires coated with Au nanoparticles were tested for hydrogen generation by combining carbon monoxide and water (the water gas shift reaction). The nanostructured catalyst used in this example is depicted in FIGS. 7A and 7B. The method to make such nanostructured catalyst has been described above as in Example 3. The reaction is:

CO+H₂O→CO₂+H₂.

The order in which the reactants were introduced into the reaction chamber was varied to test selectivity of the catalytic activity. The experiment was conducted at about room temperature and a few p.s.i. over atmosphere. The experimental setup and reaction conditions are similar to what has been described in Berven et al. IEEE Sensors J., Vol. 8 (2008), No. 6, 930, which is incorporated by reference herein in its entirety. If carbon monoxide was added to the catalyst prior to water, hydrogen was produced. In that particular instance, the nanostructured catalyst was placed in an enclosure. CO was introduced into the enclosure to interact with the catalyst. Subsequently, the CO was evacuated from the enclosure, and water was introduced. Hydrogen and carbon dioxide were observed. If the order was reversed, i.e., water was added to the catalyst prior to carbon monoxide, no hydrogen was detected. This indicates that the reaction is facilitated by the Au nanoparticles, as well as possibly by the GaN nanowire surface. The interaction between the vapor phase species and the nanoparticle catalyst could be detected by measuring the current-voltage (IV) characteristics through the GaN nanowire mat during the reaction.

This disclosure is illustrative and not limiting. Further modifications will be apparent to one skilled in the art in light of this disclosure and such modifications are intended to fall within the scope of the appended claims. Each publication and patent application cited in the specification is incorporated herein by reference in its entirety as if each individual publication or patent application were specifically and individually put forth herein.

1. A catalyst, comprising:
a disordered array of nanostructures; and
a plurality of metal-containing nanoparticles attached to the nanostructures to form a disordered array of metallized nanostructures,
wherein:
an average cross-sectional dimension of the nanoparticles is at most about half an average cross-sectional dimension of the nanostructures;
the disordered array of metallized nanostructures provides a macroporous network comprising accessible catalytic sites; and
a catalytic activity of the catalyst is tuned by i) adjusting a size of the accessible catalytic sites in the macroporous network; and ii) adjusting at least one of a metal contained in the nanoparticles and the average cross-sectional dimension of the nanoparticles.

2. The catalyst of claim 1, wherein the size of accessible catalytic sites is tuned by adjusting the configurations of the nanostructures.

3. The catalyst of claim 1, wherein the size of accessible catalytic sites is tuned by adjusting the density of the nanostructures in the disordered array.

4. The catalyst of claim 1, wherein the size of accessible catalytic sites is tuned by adjusting at least one of the configurations and density of nanostructures in the disordered array.

5. The catalyst of claim 1, wherein the size of accessible catalytic sites is tuned by adjusting at least one of the configurations and density of nanostructures in the disordered array.

6. The catalyst of claim 1, wherein a majority of the nanostructures are rod-like.

7. The catalyst of claim 1, wherein a majority of the nanostructures are coils.

8. The catalyst of claim 1, wherein the nanostructures comprise SiO₂.

9. The catalyst of claim 1, wherein the nanostructures comprise GaN.

10. The catalyst of claim 1, wherein the nanoparticles comprise a metal selected from the group consisting of Au, Ag, Pd, Pt, Fe, Ni, Co, Rh, Ru, Cu and combinations and alloys thereof.

11. The catalyst of claim 1, wherein the nanostructures comprise SiO₂ and the nanoparticles comprise Au.

12. The catalyst of claim 1, wherein the nanostructures comprise SiO₂ and the nanoparticles comprise Pt.

13. The catalyst of claim 1, wherein the nanostructures comprise SiO₂ and the nanoparticles comprise Pd.

14. The catalyst of claim 1, wherein a combination of a composition of the nanostructures and a composition of the nanoparticles is selected to tune the activity of the catalyst.

15. The catalyst of claim 1, wherein the nanostructures are coated with a metal oxide and the plurality of nanoparticles are attached to the metal oxide coating.

16. The catalyst of claim 15, wherein the metal oxide is selected from the group consisting of zinc oxide, titanium dioxide, copper oxide and cobalt oxide.

17. The catalyst of claim 1, wherein the nanoparticles comprise a metal oxide.

18. The catalyst of claim 1, where in the nanostructures consist essentially of SiO₂ and are coated with zinc oxide.

19. The catalyst of claim 18, wherein the nanoparticles comprise a metal selected from the group consisting of Au, Ag, Cu, Pd, Pt, Rh, Ru, Fe, Ni, Co and alloys and combinations thereof.

20. The catalyst of claim 1, wherein the nanoparticles are distributed on the nanostructures such that a majority of the nanoparticles are generally isolated from each other.

21. The catalyst of claim 1, wherein the nanoparticles are distributed on the nanostructures such that there is physical contact between at most about 30% of the nanoparticles.

22. A method of catalyzing a reaction, the method comprising:
selecting a size of accessible catalytic sites in a macroporous network that can adsorb one or more reactants in a catalyzed reaction, the macroporous network formed by a disordered array of nanostructures and a plurality of metal-containing nanoparticles attached to the nanostructures; and
selecting at least one of an average cross-sectional dimension of the nanoparticles and a composition of the nanoparticles to catalyze the reaction.

23. The method of claim 22, comprising selecting the shape of the size of accessible catalytic sites that can adsorb one or more reactants.

24. The method of claim 22, comprising selecting a center of the size of accessible catalytic sites that can adsorb one or more reactants.

25. The method of claim 22, comprising selecting a nanostructure configuration to determine the size of accessible catalytic sites.

26. The method of claim 22, comprising selecting a density of nanostructures in the disordered array to determine the size of accessible catalytic sites.

27. The method of claim 22, comprising selecting a combination of a composition of the nanostructures and a composition of the nanoparticles to catalyze the reaction.

28. The method of claim 22, wherein the size of the accessible catalytic sites is selected to catalyze a dehydrogenation reaction.

29. The method of claim 22, wherein the size of the accessible catalytic sites is selected to catalyze a hydrogenation reaction.

30. The method of claim 22, wherein the size of the accessible catalytic sites is selected to catalyze a polymerization reaction.

31. A catalysis device, the device comprising:
a disordered array of nanostructures, the nanostructures comprising a wide bandgap semiconductor material; and
a plurality of metal-containing nanoparticles disposed on the nanostructures to form a disordered array of metallized nanostructures providing a macroporous network with accessible catalytic sites.

32. The device of claim 31, wherein the nanostructures have a cross-sectional dimension in a range from about 5 nm to about 200 nm.

33. The device of claim 31, wherein the nanostructures comprises a semiconductor material selected from the group consisting of GaN, SiC, TiO₂, ZnO, AlN, copper oxide and cobalt oxide.

34. The device of claim 31, wherein the metal-containing nanoparticles comprise a metal selected from the group consisting of Au, Fe, Co, Ni, Cu, Rh, Ru, Pt, Pd, Ag, and combinations and alloys thereof.

35. The device of claim 31, wherein the nanoparticles have a cross-sectional dimension in a range from about 1 nm to about 5 nm.

36. A method for manufacturing a catalysis device, the method comprising:
forming a disordered array of nanostructures comprising a wide bandgap semiconductor material; and
disposing a plurality of metal-containing nanoparticles on the nanostructures to form a disordered array of metallized nanostructures to provide a macroporous network with accessible catalytic sites.

37. The method of claim 36, wherein the nanostructures have a cross-sectional dimension in a range from about 5 nm to about 200 nm.

38. The method of claim 36, wherein the semiconductor material is selected from the group consisting of GaN, SiC, TiO₂, ZnO, AlN, copper oxide and cobalt oxide.

39. The method of claim 36, wherein the nanoparticles comprise a metal selected from the group consisting of Au, Fe, Co, Ni, Cu, Rh, Ru, Pt, Pd, Ag, and combinations and alloys thereof.

40. The method of claim 36, wherein the nanoparticles have a cross-sectional dimension in a range from about 1 nm to about 5 nm.

41. A method of producing hydrogen, the method comprising:
exposing an oxygen catalysis device to CO in an enclosure, the oxygen catalysis device comprising a nanostructure comprising a wide bandgap semiconductor material and a plurality of metal-containing nanoparticles disposed on the nanostructure;
evacuating CO from the enclosure; and
after evacuating CO from the enclosure, introducing a molecule comprising hydrogen and oxygen to the oxygen catalysis device to produce hydrogen.

42. The method of claim 41, wherein the molecule comprising hydrogen and oxygen comprise H₂O so that carbon dioxide and hydrogen is produced.

43. The method of claim 41, wherein the molecule comprising hydrogen and oxygen comprises CH₃OH.

44. The method of claim 41, wherein the nanostructure has a cross-sectional dimension in a range from about 5 nm to about 200 nm.

45. The method of claim 41, wherein semiconductor material is selected from the group consisting of GaN, SiC, TiO₂, ZnO, AlN, copper oxide and cobalt oxide.

46. The method of claim 41, wherein the nanoparticles comprise a metal selected from the group consisting of Au, Fe, Co, Ni, Cu, Rh, Ru, Pt, Pd, Ag and combinations and alloys thereof.

47. The method of claim 41, wherein the nanoparticles have an average cross-sectional dimension in a range from about 1 nm to about 5 nm.

48. A method of generating hydrogen, the method comprising:
providing a nanostructured catalyst comprising metal-containing nanoparticles disposed on a substrate and/or on a disordered array of nanostructures;
reacting a compound capable of generating hydrogen and having a formula R₁—XH with the nanostructured catalyst to produce hydrogen gas and R₁—X bound to the nanostructured catalyst and/or a dehydrogenated spent compound; and
collecting the hydrogen gas,
wherein:
R₁ is a moiety selected from the group consisting of an alkyl, a heteroalkyl, an alkenyl, a substituted alkenyl, an allcynyl, an aryl, a heteroaryl, an alkoxy, a cycloalkyl, a heterocyclic, an alkylaryl, an arylalkyl, an arylalkenyl, an arylalkynyl, an arylene, an oxyarylene group, and combinations thereof; and
X is selected from the group consisting of sulfur, oxygen and selenium.

49. The method of claim 48, wherein the substrate and/or at least some of the nanostructures in the disordered array comprise a semiconductor.

50. The method of claim 48, wherein the substrate and/or at least some of the nanostructures in the disordered array comprise at least one of the group consisting of GaN, Si, SiC, TiO₂, ZnO, AN, copper oxide and cobalt oxide.

51. The method of claim 48, wherein at least some of the nanostructures in the disordered array comprise SiO₂.

52. The method of claim 51, wherein the disordered array of nanostructures comprises silica nanostructures at least partially coated with a semiconductor.

53. The method of claim 49, wherein the substrate and/or disordered array of nanostructures comprises a region comprising polycrystalline GaN.

54. The method of claim 49, wherein the substrate and/or disordered array of nanostructures comprises a region comprising single crystal GaN.

55. The method of claim 48, wherein the nanoparticles comprise a metal selected from the group consisting of Au, Ag, Cu, Pd, Pt, Ru, Rh, Fe, Ni, Co and alloys and combinations thereof.

56. The method of claim 48, wherein a surface area to mass ratio of the nanoparticles on the substrate and/or nanostructures is at least about 50 m²/g.

57. The method of claim 48, wherein disordered array comprises nanostructures having an average cross-sectional dimension in a range from about 5 nm to about 500 nm.

58. The method of claim 48, wherein the disordered array comprises nanostructures having an average cross-sectional dimension in a range from about 5 nm to about 100 nm.

59. The method of claim 48, wherein the nanostructured catalyst comprises nanoparticles having an average cross-sectional dimension in a range from about 2 nm to about 100 nm.

60. The method of claim 48, wherein the nanostructured catalyst comprises nanoparticles having an average cross-sectional dimension in a range from about 2 nm to about 15 nm.

61. The method of claim 48, wherein the nanoparticles are distributed on the substrate and/or nanostructures such that the majority of the nanoparticles are generally isolated from each other.

62. The method of claim 48, wherein the nanoparticles are distributed on the substrate and/or nanostructures such that there is physical contact between at most about 30% of the nanoparticles.

63. The method of claim 48, wherein the disordered array of nanostructures comprises nanowires, nanosprings, nanorods, nanotubes, or a combination thereof.

64. The method of claim 48, wherein the substrate and/or disordered array of nanostructures comprises GaN, and the nanoparticles disposed on the substrate and/or disordered array of nanostructures comprise Au.

65. A method of generating hydrogen, the method comprising:
providing a nanostructured catalyst comprising metal-containing nanoparticles disposed on a substrate and/or disordered array of nanostructures;
reacting a cycloalkane with the nanostructured catalyst to produce hydrogen gas and a dehydrogenated molecular species bound to the catalyst and/or a dehydrogenated spent compound; and
collecting the hydrogen gas.

66. The method of claim 65, wherein the nanoparticles comprise Pt, Pd, Ru, Rh, or a combination thereof.

67. The method of claim 65, wherein the cycloalkane is selected from the group consisting of cyclohexane, methylcyclohexane, cis-decalin, and trans-decalin.

68. The method of claim 65, wherein the disordered array comprises nanostructures having a cross-sectional dimension in a range from about 5 nm to about 100 nm.

69. The method of claim 65, wherein the nanostructured catalyst comprises nanoparticles having a cross-sectional dimension in a range from about 2 nm to about 15 nm.

70. A method for storing hydrogen for use in generating energy, the method comprising:
providing a nanostructured catalyst comprising metal-containing nanoparticles disposed on a substrate and/or disordered array of nanostructures;
reacting a dehydrogenated compound with hydrogen gas with the nanostructured catalyst to produce a compound having the formula R₁—XH; and
collecting the compound having the formula R₁—XH,
wherein:
R₁ is a moiety selected from the group consisting of an alkyl, heteroalkyl, alkenyl, substituted alkenyl, alkynyl, aryl, heteroaryl, alkoxy, cycloalkyl, heterocyclic, alkylaryl, arylalkyl, arylalkenyl, arylalkynyl, arylene, oxyarylene group, and combinations thereof; and
X is selected from the group consisting of sulfur, oxygen and selenium.

71. The method of claim 70, wherein the dehydrogenated compound is selected from the group consisting of R₁—X—X—R₁, R₂—XH, and R₃═X, wherein R₂ and R₃ are dehydrogenated relative to R₁.

72. The method of claim 70, wherein the dehydrogenated compound is selected from the group consisting of 1,2-dithiolane, dithioparabenzoquinone, and 1,4-benzenedithiol.

73. The method of claim 70, wherein the disordered array comprises nanostructures having a cross-sectional dimension in a range from about 5 nm to about 100 nm.

74. The method of claim 70, wherein the nanostructured catalyst comprises nanoparticles having a cross-sectional dimension in a range from about 2 nm to about 15 nm.

75. A method for storing hydrogen for use in generating energy, the method comprising:
providing a nanostructured catalyst comprising metal-containing nanoparticles disposed on a substrate and/or a disordered array of nanostructures;
reacting a dehydrogenated compound with hydrogen gas with the nanostructured catalyst to produce a cycloalkane; and
collecting the cycloalkane.

76. The method of claim 75, comprising producing cyclohexane, methylcyclohexane, cis-decalin, or trans-decalin.

77. The method of claim 75, wherein the disordered array comprises nanostructures having a cross-sectional dimension in a range from about 5 nm to about 100 nm.

78. The method of claim 75, wherein the nanostructured catalyst comprises nanoparticles having a cross-sectional dimension in a range from about 2 nm to about 15 nm

79. The method of claim 65, wherein collecting the hydrogen gas comprises consuming the hydrogen gas in an engine or fuel cell.

80. The method of claim 48, comprising reacting the nanostructured catalyst with a compound having the formula R₁—SH.

81. The method of claim 48, wherein R₁ is a C2-C8 alkyul, heteroalkly, alkenyl, or heteroalkenyl group.

82. The method of claim 48, comprising reacting the nanostructured catalyst with a C2-C8 organothiol.

83. The method of claim 48, comprising reacting the nanostructured catalyst with 1,4-cyclohexanedithiol.

84. The method of claim 48, wherein collecting the hydrogen gas comprises consuming the hydrogen gas in an engine or fuel cell.
